SEQUENCE LISTING (21 sequences)

SEQ ID NO: 1 - DNA, 2797 bp, D. melanogaster
gttcattaaaatatgtggtgataacgcgagctgccgaatctgcgtgcaattcgtgcgtttgacgtgggtactaactgctatgctgtcgcgcggacagttgttctgatacgcagagttcctgcctcaccacacacgaccacctccattaaaaccagccaccccccccagcgcctcctccaccgacagcagctgctccaccgcaccaccaggagaggggcaattaaaaaatcaatcagagggccctaattgaaagctgccaccgtcgaaatgtcgccgccgaagaactgcgcggtgtgcggggacaaggctctgggctacaacttcaatgcggtcacctgcgagagctgcaaggcgttcttccgacggaacgcgctggccaagaagcagttcacctgccccttcaaccaaaactgcgacatcactgtggtcactcgacgcttctgccagaaatgccgcctgcgcaagtgcctggatatcgggatgaagagtgaaaacattatgtccgaggaggacaagctgatcaagcggcgcaagatcgagaccaaccgggccaagcgacgcctcatggagaacggcacggatgcgtgcgacgccgatggcggcgaggaaagggatcacaaagcgccggcggatagcagcagcagcaaccttgaccactactcggggtcacaggactcgcagagctgcggctcggcggacagcggggccaatgggtgctccggcagacaggccagttcgccgggcacacaggtcaatccgcttcagatgacggccgagaagatagtcgaccagatcgtatccgacccggatcgagcctcgcaggccatcaaccggttgatgcgcacgcagaaagaggctatatcggtgatggagaaggtaatcagctcacaaaaggacgccttaaggctggtgtcgcatttgatcgactatccaggcgacgcactcaagatcatttcaaagtttatgaactcgccctttaacgcgctgacagtattcaccaaattcatgagctcacccacggacggcgttgaaattatctcaaagatagttgattcgcccgcggacgtggtggagttcatgcagaacttgatgcactcgccagaggacgccatcgatataatgaacaagttcatgaataccccagcggaggcgctgcgcattcttaaccgaatcctaagcggcggaggagcgaacgcagcccagcagacagcagaccgcaagccattgctggacaaggagccggcggtgaagcctgcagcgccagcggagcgagctgatactgtcattcaaagcatgctgggcaacagtccgccaatttcgccacatgatgctgccgtggatctgcagtaccactcgcccggtgtcggggagcagcccagtacatcgagtagccaccccttgccttacatagccaactcgccggacttcgatctgaagaccttcatgcagaccaactacaacgacgagcccagtctggacagtgattttagcattaactcaatcgaatcggtgctatccgaggtgatccgcattgagtaccaggccttcaatagcatacaacaagcggcatcgcgcgtaaaggaggagatgtcctacggcactcagtctacgtacggtggatgcaattcggctgcaaacaatagccagccgcacctgcagcaacccatctgcgccccatccacccagcagttggatcgcgagctaaacgaggcggagcaaatgaagctgcgggagctgcgactggccagcgaggctctttatgatcccgtggacgaggacctcagcgccctgatgatgggcgatgatcgcattaagcccgacgacactcgccacaacccaaagctattgcagctgatcaatctgacggcggtggccatcaagcggcttatcaaaatggccaagaagattacagcattccgtgacatgtgccaggaggaccaggtggccctactcaaaggtggctgcacagaaatgatgataatgcgctccgtaatgatttacgacgacgatcgcgccgcctggaaggtaccccataccaaagagaacatgggcaacatacgcactgacctgctcaagtttgccgaaggcaatatctacgaggagcaccaaaagttcatcacaacgtttgacgagaagtggcgcatggacgagaacataatcctgatcatgtgtgccattgtcctttttacctcggctcgatcgcgagtgatacacaaagacgtgattagattggaacagaattcctactattatcttctgcgaagatatctggagagtgtttattctggctgtgaggcgagaaacgcgtttatcaagctaatccaaaagatttcagatgtggagcgtctgaacaagttcataattaatgtctatttgaatgttaacccatcccaggtggagcccttgctgcgtgaaatattcgatttgaaaaatcactagacaaccgatgcgtgtcgggcatttaatgcctatgttgatgcccaatgatgaatggtcaacaagctgtagttgttgttgttgttgatgtctgttttatcttgtcgcttgtaatgttagattttaatcgaatgtgattgttagatttgcatatactgcatagattttatatttctacatcaaagagagcatatttaggataccaagtgcaaagcaacacaatctatatgtaatgtacaccgtttacctagtttcaaataaactagacgataatgcaataactaacttggaagcgtgggttctgtgcaaaaaggaaaaaagacaaaaaaaataaactgactttgagaaccagtggtaaacc

SEQ ID NO: 2 - DNA, 36 bp, Artificial sequence
  Primer dhr96-F
tattgcggatccttgacgtgggtactaactgctatg

SEQ ID NO: 3 - DNA, 34 bp, Artificial sequence
  Primer dhr96-R
agtccggaattcagtagtggtcaaggttgctgct

SEQ ID NO: 4 - DNA, 24 bp, Artificial sequence
  dhr96-fwd_primer
catggacgagaacataatcctgat

SEQ ID NO: 5 - DNA, 30 bp, Artificial sequence
  dhr96-rev_primer
cagaagataatagtaggaattctgttccaa

SEQ ID NO: 6 - DNA, 27 bp, Artificial sequence
  dhr96-taqman_probe
tgtgccattgtcctttttacctcggct

SEQ ID NO: 7 - DNA, 24 bp, Artificial sequence
  RpL32-fwd_primer
gatatgctaagctgtcgcacaaat

SEQ ID NO: 8 - DNA, 23 bp, Artificial sequence
  RpL32-rev_primer
ggcatcagatactgtcccttgaa

SEQ ID NO: 9 - DNA, 24 bp, Artificial sequence
  RpL32-taqman_probe
cgcaagcccaagggtatcgacaac

SEQ ID NO: 10 - protein, 263 aa, Artificial sequence
  DHR96_peptide2
TDGVEIISKIVDSPADVVEFMQNLMHSPEDAIDIMNKFMNTPAEALRILNRILSGGGANAAQQTADRKPLLDKEPAVKPAAPAERADTVIQSMLGNSPPISPHDAAVDLQYHSPGVGEQPSTSSSHPLPYIANSPDFDLKTFMQTNYNDEPSLDSDFSINSIESVLSEVIRIEYQAFNSIQQAASRVKEEMSYGTQSTYGGCNSAANNSQPHLQQPICAPSTQQLDRELNEAEQMKLRELRLASEALYDPVDEDLSALMMGDD

SEQ ID NO: 11 - DNA, 440 bp, D. melanogaster
atcccaaaacaaactggttattgtggtaggtcatttgtttggcagaaagaaaactcgagaaatttctctggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaaga

SEQ ID NO: 12 - DNA, 441 bp, S. cerevisiae
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcg

SEQ ID NO: 13 - protein, 147 aa, S. cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 14 - DNA, 1944 bp, D. melanogaster
aacattatgtccgaggaggacaagctgatcaagcggcgcaagatcgagaccaaccgggccaagcgacgcctcatggagaacggcacggatgcgtgcgacgccgatggcggcgaggaaagggatcacaaagcgccggcggatagcagcagcagcaaccttgaccactactcggggtcacaggactcgcagagctgcggctcggcggacagcggggccaatgggtgctccggcagacaggccagttcgccgggcacacaggtcaatccgcttcagatgacggccgagaagatagtcgaccagatcgtatccgacccggatcgagcctcgcaggccatcaaccggttgatgcgcacgcagaaagaggctatatcggtgatggagaaggtaatcagctcacaaaaggacgccttaaggctggtgtcgcatttgatcgactatccaggcgacgcactcaagatcatttcaaagtttatgaactcgccctttaacgcgctgacagtattcaccaaattcatgagctcacccacggacggcgttgaaattatctcaaagatagttgattcgcccgcggacgtggtggagttcatgcagaacttgatgcactcgccagaggacgccatcgatataatgaacaagttcatgaataccccagcggaggcgctgcgcattcttaaccgaatcctaagcggcggaggagcgaacgcagcccagcagacagcagaccgcaagccattgctggacaaggagccggcggtgaagcctgcagcgccagcggagcgagctgatactgtcattcaaagcatgctgggcaacagtccgccaatttcgccacatgatgctgccgtggatctgcagtaccactcgcccggtgtcggggagcagcccagtacatcgagtagccaccccttgccttacatagccaactcgccggacttcgatctgaagaccttcatgcagaccaactacaacgacgagcccagtctggacagtgattttagcattaactcaatcgaatcggtgctatccgaggtgatccgcattgagtaccaggccttcaatagcatacaacaagcggcatcgcgcgtaaaggaggagatgtcctacggcactcagtctacgtacggtggatgcaattcggctgcaaacaatagccagccgcacctgcagcaacccatctgcgccccatccacccagcagttggatcgcgagctaaacgaggcggagcaaatgaagctgcgggagctgcgactggccagcgaggctctttatgatcccgtggacgaggacctcagcgccctgatgatgggcgatgatcgcattaagcccgacgacactcgccacaacccaaagctattgcagctgatcaatctgacggcggtggccatcaagcggcttatcaaaatggccaagaagattacagcattccgtgacatgtgccaggaggaccaggtggccctactcaaaggtggctgcacagaaatgatgataatgcgctccgtaatgatttacgacgacgatcgcgccgcctggaaggtaccccataccaaagagaacatgggcaacatacgcactgacctgctcaagtttgccgaaggcaatatctacgaggagcaccaaaagttcatcacaacgtttgacgagaagtggcgcatggacgagaacataatcctgatcatgtgtgccattgtcctttttacctcggctcgatcgcgagtgatacacaaagacgtgattagattggaacagaattcctactattatcttctgcgaagatatctggagagtgtttattctggctgtgaggcgagaaacgcgtttatcaagctaatccaaaagatttcagatgtggagcgtctgaacaagttcataattaatgtctatttgaatgttaacccatcccaggtggagcccttgctgcgtgaaatattcgatttgaaaaatcac

SEQ ID NO: 15 - protein, 648 aa, D. melanogaster
NIMSEEDKLIKRRKIETNRAKRRLMENGTDACDADGGEERDHKAPADSSSSNLDHYSGSQDSQSCGSADSGANGCSGRQASSPGTQVNPLQMTAEKIVDQIVSDPDRASQAINRLMRTQKEAISVMEKVISSQKDALRLVSHLIDYPGDALKIISKFMNSPFNALTVFTKFMSSPTDGVEIISKIVDSPADVVEFMQNLMHSPEDAIDIMNKFMNTPAEALRILNRILSGGGANAAQQTADRKPLLDKEPAVKPAAPAERADTVIQSMLGNSPPISPHDAAVDLQYHSPGVGEQPSTSSSHPLPYIANSPDFDLKTFMQTNYNDEPSLDSDFSINSIESVLSEVIRIEYQAFNSIQQAASRVKEEMSYGTQSTYGGCNSAANNSQPHLQQPICAPSTQQLDRELNEAEQMKLRELRLASEALYDPVDEDLSALMMGDDRIKPDDTRHNPKLLQLINLTAVAIKRLIKMAKKITAFRDMCQEDQVALLKGGCTEMMIMRSVMIYDDDRAAWKVPHTKENMGNIRTDLLKFAEGNIYEEHQKFITTFDEKWRMDENIILIMCAIVLFTSARSRVIHKDVIRLEQNSYYYLLRRYLESVYSGCEARNAFIKLIQKISDVERLNKFIINVYLNVNPSQVEPLLREIFDLKNH

SEQ ID NO: 16 - protein, 229 aa, Anemonia majano
MALSNKFIGDDMKMTYHMDGCVNGHYFTVKGEGNGKPYEGTQTSTFKVTMANGGPLAFSFDILSTVFKYGNRCFTAYPTSMPDYFKQAFPDGMSYERTFTYEDGGVATASWEISLKGNCFEHKSTFHGVNFPADGPVMAKKTTGWDPSFEKMTVCDGILKGDVTAFLMLQGGGNYRCQFHTSYKTKKPVTMPPNHVVEHRIARTDLDKGGNSVQLTEHAVAHITSVVPF

SEQ ID NO: 17 - protein, 232 aa, Anemonia sulcata
MASFLKKTMPFKTTIEGTVNGHYFKCTGKGEGNPFEGTQEMKIEVIEGGPLPFAFHILSTSCMYGSKTFIKYVSGIPDYFKQSFPEGFTWERTTTYEDGGFLTAHQDTSLDGDCLVYKVKILGNNFPADGPVMQNKAGRWEPATEIVYEVDGVLRGQSLMALKCPGGRHLTCHLHTTYRSKKPASALKMPGFHFEDHRIEIMEEVEKGKCYKQYEAAVGRYCDAAPSKLGHN

SEQ ID NO: 18 - protein, 231 aa, Zoanthus sp.
MAHSKHGLKEEMTMKYHMEGCVNGHKFVITGEGIGYPFKGKQTINLCVIEGGPLPFSEDILSAGFKYGDRIFTEYPQDIVDYFKNSCPAGYTWGRSFLFEDGAVCICNVDITVSVKENCIYHKSIFNGMNFPADGPVMKKMTTNWEASCEKIMPVPKQGILKGDVSMYLLLKDGGRYRCQFDTVYKAKSVPSKMPEWHFIQHKLLREDRSDAKNQKWQLTEHAIAFPSALA

SEQ ID NO: 19 - protein, 231 aa, Zoanthus sp.
MAQSKHGLTKEMTMKYRMEGCVDGHKFVITGEGIGYPFKGKQAINLCVVEGGPLPFAEDILSAAFNYGNRVFTEYPQDIVDYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 20 - DNA, 30 bp, Artificial sequence
  Forward degenerate primer
  misc_feature (3)..(21): N at each of positions 3, 6, 9, 12, 15 and 21 represents independently any one of A, C, T or G
ytnytnaarggnggntgyacngaratgatg

SEQ ID NO: 21 - DNA, 30 bp, Artificial sequence
  Reverse degenerate primer
  misc_feature (1)..(7): N at each of positions 1, 4 and 7 represents independently any one of A, C, T or G.
ncknarnarrtartartarctrttytgytc

(19) United States
(12) Patent Application Publication
(10) Pub. No.: US 2009/0031433 A1
(43) Pub. Date: Jan. 29, 2009

(54) METHODS FOR SCREENING INSECTICIDES

(75) Inventors: Sheetal Mansukhlal Shah, Berkshire (GB); John David Windass, Berks (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA LIMITED, Guildford, Surrey (GB)

(21) Appl. No.: 10/597,719

(22) PCT Filed: Feb. 17, 2005
(86) PCT No.: PCT/GB05/00591

§ 371 Date: Apr. 30, 2007

(30) Foreign Application Priority Data
Feb. 23, 2004 (GB) 0403966.5

(51) Int. Cl. C12N 15/12; A01K 67/033; G01N 33/50

(52) U.S. Cl. 800/3; 800/13; 536/23.5; 800/21

(57) ABSTRACT

The present invention relates to methods of screening for potential insecticidal agents using genes encoding proteins that regulate xenobiotic detoxification in insects. In particular the invention relates to methods of using insect nuclear hormone receptor genes, in particular dhr96 or homologues thereof and/or their promoters. Such methods employ inter alia transgenic cells and/or transgenic whole organisms in which one or more nuclear hormone receptor gene is deleted or under-expressed.

The present invention relates to methods of screening for potential insecticidal agents using genes encoding proteins that act as xenobiotic sensors and which regulate a molecular response such as xenobiotic detoxification or metabolism in insects. In particular the invention relates to methods of using insect nuclear hormone receptor genes and/or their promoters, especially the Drosophila dhr96 gene and/or promoter and homologues thereof, as well as to the products of such insect nuclear hormone receptor genes, to identify and/or optimise potential insecticidal agents. The methods employ transgenic cells and/or whole organisms in which an insect nuclear hormone receptor gene is deleted or under-expressed as well as transgenic cells, cell-lines and/or organisms that over-express an insect nuclear hormone receptor gene or express an insect nuclear horomone receptor gene in a heterologous manner. The invention further extends to the transgenic cells, cell-lines and/or organisms used in the methods of the invention as well as to DNA constructs comprising an insect nuclear hormone receptor gene or a fragment thereof and DNA constructs comprising the promoter of an insect nuclear hormone receptor gene.

Different classes of xenobiotic compounds have been, and are being, used as insecticides. However, resistance to a large number of these compounds has emerged in most insect species. Resistance to xenobiotic compounds may be mediated by one or more different molecular mechanisms e.g. target site alteration (see for example: Mutero et al., 1994, Proc. Natl. Acad. Sci. 91:5922-5926; ffrench-Constant et al., 2000, Annu. Rev. Entomol. 45:449-466; Williamson et al., 1996, Molec. Gen. Genet 252:51-60), enzymatic modification or degradation of the xenobiotic (e.g. by P450s, carboxylesterases, glutathione S-transferases; Ranson et al., 2002, Science 298:179-181), and active efflux (Foote et al., 1990, Nature 345:255-258; Lanning et al., 1996, Toxicol Lett. 85:127-133).

Accordingly there exists a need to develop novel insecticides that are capable of by-passing one or more of the above-mentioned molecular mechanisms of resistance.

One conventional method of identifying novel insecticidal compounds relies on screening compounds against wild-type insects and assessing whether the compound has any deleterious effect on the insect. When potential insecticidal compounds are screened for activity against wild-type insects, only very few are shown to be highly insecticidally active and it is routinely only these compounds that are taken forward into further development as a potential insecticide. The vast majority of compounds, which either exhibit only a low level of insecticidal activity or which have no insecticidal activity at all, are often discarded.

The apparent lack of insecticidal activity of a compound screened against wild-type insects may be due to the fact that the compound is truly incapable of acting as an insecticide. Alternatively, the apparent lack of insecticidal activity may be due to the compound being detoxified or metabolised by the insect to form a less potent compound, for example via P450 or other enzyme mediated metabolism, or as a result of active efflux of the compound from insect cells.

It is thus likely that some of the compounds that are routinely discarded, due to their lack of insecticidal activity against wild-type insects, have the potential to be insecticides if they were modified so that they were no longer susceptible to such xenobiotic detoxification mechanisms.

This disadvantage of the conventional method of screening (i.e. the fact that weak chemical leads are often discarded) is addressed by the current invention which uses transgenic insects and transgenic insect cells wherein a xenobiotic detoxification pathway has been altered such that xenobiotic detoxification in the transgenic insect or insect cell is reduced relative to xenobiotic detoxification in a wild-type insect or insect cell and these transgenic insects and/or transgenic insect cells are thus used to screen for, and identify, weak chemical leads.

The present invention achieves this reduction in xenobiotic detoxification in an indirect manner by reducing the level of expression of one or more key genes that act as a “master-switch” in regulating detoxification (e.g. P450-mediated or other xenobiotic detoxification) pathways in insects. These key genes act upstream of, for example, P450 or cyp gene(s) in a P450 metabolic pathway and are thought to act as regulators of cyp gene expression. Thus by reducing the level of expression or activity of such a key “master-switch” gene, the level of expression of one or more downstream (e.g. cyp) genes is indirectly reduced. The present invention is based on the finding that the nuclear hormone receptor gene dhr96 from Drosophila melanogaster is one such “master-switch”.

Thus in a first aspect of the invention there is provided a transgenic insect or transgenic insect cell wherein the level of expression of an insect nuclear hormone receptor gene has been reduced relative to the level of expression of said nuclear hormone receptor gene in a non-transgenic insect or insect cell.

For the avoidance of doubt the term “transgenic” as used herein in reference to an insect or insect cell, describes an insect or an insect cell wherein the genetic material of said insect or insect cell has been altered using recombinant DNA technology. Thus, a transgenic insect or insect cell of the invention does not include a naturally occurring or chemically produced mutant insect or insect cell wherein the level of expression of a nuclear hormone receptor gene has been reduced relative to the level of expression of said nuclear hormone receptor gene in a non-mutant or wild-type insect or insect cell.

The nuclear hormone receptor gene whose level of expression is reduced in a transgenic insect or insect cell of the invention is preferably the dhr96 gene from D. melanogaster or a homologue of dhr96.

The term “homologue” as used herein with respect to an insect nuclear hormone receptor gene refers to any gene which functions in the same way (i.e. which acts as a xenobiotic sensor and thus, for example, regulates xenobiotic detoxification or metabolism) as an insect nuclear hormone receptor gene and/or which exhibits substantial identity to a named insect nuclear hormone receptor gene. Typically a homologue of the invention will be an insect nuclear hormone receptor gene that classifies into the NR1 family of nuclear hormone receptors (Maglich et al. 2001, Genome Biology 2:research 0029.1-0029.7), and especially into the 1H, 1I and 1J sub-families (Cell, 1999, 97:161-163). Preferably a homologue of the invention encodes a protein which exhibits at least 60%, 65%, 70%, 75%, 80%, 85%, 90%, or 95% identity with a nuclear hormone receptor encoded by a named insect nuclear hormone receptor gene. Most preferably a homologue encodes a protein that exhibits at least 96%, 97%, 98% or 99% identity with that encoded by a named insect nuclear hormone receptor gene. In one embodiment a homologue will exhibit at least 80% identity in the DNA binding domain and at least 40% identity in the ligand binding domain in comparison to said domains from a named insect nuclear hormone receptor. In one embodiment the named insect nuclear hormone receptor gene is dhr96 from D. melanogaster.

Homologues include nuclear hormone receptor genes found in the same insect genus and species as the named nuclear hormone receptor gene, as well as nuclear hormone receptor genes from insects of other genera and/or species. Thus the homologue may come from the group of insects consisting of: Drosophila sp. Bombyx ori, Tribolium castaneum, Aedes aegyptii, Anopheles gambiae, Anopheles albimanus, Anopheles stephensi, Ceratitis capitata, Pectinophora gossypiella, Helicoverpa zea, Bactrocera dorsalis, Anastrepha suspense, Musca domestica, Stomoxys calcitrans, Heliothis virescens, Manduca sexta and Lucilia cuprina. Prefereably the homologue will come from a known insect pest species, including in particular (but not limited to) Heliothis virescens and Manduca sexta.

The transgenic insect or transgenic insect cell may be any insect or insect cell which is capable of being transformed. In particular the transgenic insect may be, or the transgenic insect cell may come from, any one of the following insects: Drosophila sp. Bombyx mori, Tribolium castaneum, Aedes aegyptii, Anopheles gambiae, Anopheles albimanus, Anopheles stephensi, Ceratitis capitata, Pectinophora gossypiella,, Helicoverpa zea, Bactrocera dorsalis, Anastrepha suspense, Musca domestica, Stomoxys calcitrans, and Lucilia cuprina. In addition, a transgenic insect cell of the invention may also be derived from Spodoptera frugiperda or Trichoplusia ni. In one embodiment it is particularly preferred that the transgenic insect cell is derived from a D. melanogaster S2 cell.

Insects from the genus Drosophila, in particular Drosophila melanogaster, Drosophila simulans and Drosophila virilis are considered as particularly suitable for use in the invention. In one preferred embodiment, transgenic insects or insect cells of the invention are derived from D. melanogaster.

Transgenic insects and insect cells according to this aspect of the invention exhibit a reduced level of expression of an insect nuclear hormone receptor gene in comparison to the level of expression of the nuclear hormone receptor gene in a wild-type non-transgenic insect or insect cell. Where the transgenic insect is D. melanogaster, D. melanogaster Canton S is particularly suitable as a wild-type non-transgenic insect and where the transgenic insect cell is derived from a Drosophila cell line, such as the Drosophila S2 cell line, the untransformed cell line may act as a suitable non-transgenic insect cell for the purposes of comparison.

For the avoidance of doubt, a reduced level of expression of a nuclear hormone receptor gene includes any level of expression that is less than the level of expression of the nuclear hormone receptor gene in the non-transgenic insect or insect cell and includes no detectable expression of said nuclear hormone receptor gene in the transgenic insect or insect cell.

The reduced level of expression of the nuclear hormone receptor gene in the transgenic insect or insect cell may be effected through gene disruption at the locus for the nuclear hormone receptor gene in the insect concerned. This may achieved through homologous recombination (see Rong & Golic 2000 Science 288(5473):2013-2018 for methodology relating to homologous recombination in Drosophila) or through insertional mutagenesis e.g. transposon insertion.

Alternatively the reduced level of nuclear hormone receptor expression may be effected using an RNA-interference (RNAi) mediated knockdown or knockout of nuclear hormone receptor gene function, via either a transient RNAi approach, or via the generation of a stable transgenic insect line (Kennerdell & Carthew 2000, Nature Biotech. 17:896-898. Where a stable Drosophila line is produced, it is preferred that the reduction in nuclear hormone receptor gene expression level is inducible. This may be achieved by employing the GAL4-UAS system (Brand & Perrimon, 1993, Development 118:401-415) as described herein in the Examples.

In one embodiment of the invention where the reduction in the level of nuclear hormone receptor gene expression is mediated through an RNAi approach, an insect or insect cell is transformed with a DNA construct comprising a first DNA sequence encoding a fragment of an insect nuclear hormone receptor gene and a second DNA sequence which is the reverse complement of the first DNA sequence, wherein the first and second DNA sequences are present on the same strand of DNA. Upon transcription a mRNA molecule will be produced that is capable of forming a hairpin or stem-loop structure by virtue of the complementarity of the regions in the mRNA which correspond to the first and second DNA sequences. This double stranded mRNA region acts as a source of short interfering RNA molecules and thus mediates the knockdown or knockout of nuclear hormone receptor gene function, which is manifested in transgenic insects or insect cells as a reduced level of expression of the nuclear hormone receptor gene.

Thus in a further aspect the invention provides a DNA construct comprising a first DNA sequence encoding a fragment of an insect nuclear hormone receptor gene and a second DNA sequence which is the reverse complement of the first DNA sequence, wherein the first and second DNA sequences are present on the same strand of DNA and are operably linked to a promoter region and optionally a terminator region.

It is thought that in order for the level of gene expression to be reduced through an RNAi mediated mechanism, the minimum size of a short interfering RNA is between 21 and 23 nucleotides (Zamore et al. 2000 Cell 101:25-33). Accordingly for just a single short interfering RNA to be produced from the transcript of a DNA construct of this aspect of the invention, the minimum length of the first and second DNA sequences in said DNA construct is 23 nucleotides. However, it is preferred that the length of the first and second DNA sequences is considerably longer than 23 nucleotides. Preferably the first and second DNA sequences will be greater than or equal to 100, 200, 300, 400, 500, 600, 700, 800, 900, 1000, or 1500 nucleotides in length.

In one embodiment a DNA construct of the invention will additionally comprise a region of DNA which acts as a spacer region between the first and second DNA sequences. Such a DNA sequence may be incorporated to facilitate cloning or to facilitate the formation of a double-stranded mRNA structure, such as a hairpin or stem-loop structure, when the DNA sequence of the DNA construct is transcribed.

Sequences that are known to be introns in a gene of the insect that is to be transformed with a DNA construct of the invention are particularly suitable for this purpose.

The first and second DNA sequences that are present in a DNA construct of the invention are operably linked to a promoter region. In general the promoter region will be any promoter region capable of driving expression of the first and second DNA sequences in the insect or insect cell into which a DNA construct of the invention is to be introduced. The promoter region may comprise (or consist of) a constitutive promoter, a tissue- or developmentally-specific promoter, or an inducible promoter. Preferably the promoter region comprises (or consists of) an inducible promoter so that the reduction in the level of expression of the nuclear hormone receptor gene is controllable. Preferably the inducible promoter comprises UAS hsp70 TATA promoter sequences responsive to the GAL4 protein.

DNA constructs of the invention optionally comprise a transcriptional terminator region. Typically, such a transcriptional terminator region will be one that is suitable for the termination transcription in the insect/insect cell into which the DNA construct is to be introduced. Transcriptional terminator regions suitable for this purpose are described in the art.

DNA constructs of the invention are introduced into insect cells to form transgenic insects and insect cells using any suitable method available in the art. In particular, D. melanogaster embryos may be transformed using the methodology described by Spradling & Rubin (1982, Science 218:341-347), for example, as described herein in the Examples.

Where the promoter region of a DNA construct of the invention comprises or consists of a constitutive promoter region, transformation of the DNA construct into an insect or insect cell will yield a transgenic insect or insect cell of the invention. Thus, a transgenic insect or insect cell of the invention may be produced merely by transforming an appropriate insect or insect cell with a DNA construct as described hereinbefore.

However, where the promoter region comprises or consists of an inducible promoter an inducing agent must be applied to (or be present within) the transformed insect or insect cell.

In Drosophila this may be achieved through the use of a binary system as described by Brand & Perrimon (1993, Development 118:401-415) and as used herein in the Examples. Practically, a DNA construct of the invention is introduced into a first insect and a second DNA construct, which encodes a protein capable of regulating expression from the inducible promoter in the DNA construct of the invention, is introduced into a second insect. The first and second insects are then genetically crossed to yield progeny, a proportion of which will comprise both the first and second DNA constructs. Where expression of the protein encoded by the second DNA construct is under the control of a constitutive promoter, the population of progeny comprising both the first and second DNA constructs will comprise not only a DNA construct of the invention but also the inducing agent necessary to effect induction from the promoter region in the first DNA construct. Consequently the population of progeny, which comprises both the first and second DNA constructs, consists of transgenic insects of the invention.

Thus in a further aspect of the invention there is provided a method of producing a transgenic insect of the invention, which comprises: (i) transforming a first insect with a first DNA construct as described hereinbefore wherein the promoter region comprises an inducible promoter; (ii) transforming a second insect with a second DNA construct encoding a protein that is capable of inducing expression from the inducible promoter in the first DNA construct; (iii) crossing the transformed insects resulting from steps (i) and (ii) to obtain progeny; wherein a population of the progeny resulting from step (iii) comprises both the first and the second DNA constructs and when the protein encoded in the second DNA construct is expressed, the level of expression of the nuclear hormone receptor gene is reduced in said population of the progeny relative to the level of expression of the nuclear hormone receptor gene in a non-transgenic insect.

Insects containing the second DNA construct (e.g. insects already transformed with a DNA construct encoding a protein that is capable of inducing expression from the inducible promoter in the first DNA construct) are also readily available in the art (see Example 4 hereinafter). Accordingly the invention also provides a method of producing a transgenic insect of the invention, which comprises: (i) transforming a first insect with a first DNA construct as described hereinbefore wherein the promoter region comprises an inducible promoter; (ii) crossing the transformed insect resulting from step (i) with an insect that expresses an inducing protein that is capable of inducing expression from the inducible promoter in the first DNA construct, to obtain progeny; wherein a population of the progeny resulting from step (ii) comprise the first DNA construct and express the inducing protein so that the level of expression of the nuclear hormone receptor gene is reduced in said population of the progeny relative to the level of expression of the nuclear hormone receptor gene in a non-transgenic insect.

In a similar manner, a transgenic insect cell may be made by co-transforming a suitable insect cell with a DNA construct of the invention and a second DNA construct that encodes a protein capable of regulating expression from the inducible promoter in the DNA construct of the invention. The skilled man will also appreciate that a transgenic insect cell may be made by incorporating the features of both DNA constructs into a single construct which is then transformed into the cell.

The transgenic insects and transgenic insect cells of the invention are more susceptible to compounds that are subject to xenobiotic metabolism wherein the metabolic pathway is regulated by the insect nuclear hormone receptor gene whose expression level has been reduced therein. Thus the transgenic insects and insect cells of the invention are useful in identifying compounds which are potential insecticides and which would not have been identified, or would have only been identified as being weakly active, in a conventional screen using a wild-type and/or non-transgenic insect or non-transgenic insect cell.

Thus in a further aspect of the invention there is provided a method of identifying or verifying the ability of a compound to act as an insecticide, which comprises: (i) placing a transgenic insect or transgenic insect cell of the invention in contact with the compound; (ii) assessing the transgenic insect or transgenic insect cell that has been placed in contact with the compound in step (i) for any deleterious effect on the insect or insect cell; wherein the presence of a deleterious effect is indicative that said compound is capable of acting as an insecticide.

A deleterious effect may be manifested as the death of the insect or insect cells, or by slow growth of the insect or insect cell in comparison to insect or insect cells that have not been treated with the compound, or by some other clear phenotypic change in treated insects or insect cells in comparison to untreated insects or insect cells.

The present invention also provides assays for identifying insecticidal compounds that are not susceptible to detoxification via a pathway regulated via an insect nuclear hormone receptor (e.g. dhr96 or a homologue thereof), in particular compounds that are resistant to degradation by the products of insect cyp gene or genes that are regulated by an insect nuclear hormone receptor. For example the effect of a test compound on a transgenic insect or insect cell wherein the level of the nuclear hormone receptor (e.g. dhr96 or a homologue thereof) is reduced can be compared to the effect of the same compound on non-transgenic control insects or non-transgenic insect cells, and/or compared to the effect of the same compound on transgenic insects or transgenic insect cells wherein the level of nuclear hormone receptor has been amplified (increased) above that observed in non-transgenic insects or non-transgenic insect cells. If such a comparison reveals no significant difference in the effects observed on all of the insects/insect cells assayed, this would be indicative that the test compound is not susceptible to detoxification by any pathway regulated by the nuclear hormone receptor. Further, if a deleterious effect is noted on all of the insects treated with the test compound (in comparison to those treated with no test compound as a control), this will indicate that the test compound is capable of acting insecticidally.

The invention also provides a method of reducing the susceptibility of a compound to detoxification by a pathway that is regulated by an insect nuclear hormone receptor.

In yet a further aspect, the invention provides assays for identifying pro-insecticidal compounds, which require in vivo conversion to an active pesticide and wherein such conversion is mediated through a metabolic pathway regulated by DHR96 or a homologue thereof. In transgenic insects or transgenic insect cells wherein the level of DHR96 (or corresponding homologue) has been reduced relative to non-transgenic insects or non-transgenic insect cells, it is thought that the metabolic pathways regulated by DHR96 (or the corresponding homologue) will no-longer be activated. As a consequence, any pro-insecticide relying on one of the pathways regulated by DHR96 (or corresponding homologue) for conversion to an active insecticide will not undergo conversion when applied to such transgenic insects or transgenic insect cells. Thus transgenic insects or transgenic insect cells according to the first aspect of the invention will appear to have greater resistance to the putative pro-insecticidal compound in comparison to non-transgenic control insects or non-transgenic control insect cells.

In addition to utilising transgenic insect and transgenic insect cells as described hereinbefore, these assays may additionally employ cells and/or whole organisms that over-express an insect nuclear hormone receptor gene, or which express an insect nuclear hormone receptor gene in a heterologous manner.

Where these assays involve the use of cells and/or whole organisms that over-express an insect nuclear hormone receptor gene, or which express an insect nuclear hormone receptor gene in a heterologous manner such cells and/or whole organisms will comprise a DNA construct comprising the dhr96 gene from Drosophila melanogaster, or a homologue thereof, operably linked to a promoter region and optionally a transcriptional terminator. Such DNA constructs are referred to herein as “expression constructs”, and form yet a further aspect of the invention. It is preferred that the nuclear hormone receptor gene will be selected from the group consisting of dhr96 from D. melanogaster and homologues thereof derived from the following insects: Drosophila sp. Bombyx ori, Tribolium castaneum, Aedes aegyptii, Anopheles gambiae, Anopheles albimanus, Anopheles stephensi, Ceratitis capitata, Pectinophora gossypiella, Helicoverpa zea, Bactrocera dorsalis, Anastrepha suspense, Musca domestica, Stomoxys calcitrans, Heliothis virescens, Manduca sexta and Lucilia cuprina. It is particularly preferred that nuclear the hormone receptor gene is dhr96 from D. melanogaster.

In general the promoter region to which the insect nuclear hormone receptor gene is operably linked will be any promoter region capable of driving expression of the insect nuclear hormone receptor gene in the host cell into which an expression construct comprising the insect nuclear hormone receptor gene is to be introduced. Thus, if it is desired that the expression construct be introduced into a bacterial cell, the promoter will be operable in that bacterial cell. Similarly if it is desired that the expression construct be introduced into a yeast cell, the promoter will be operable in that yeast cell and the same logic prevails if the construct is to be introduced into an insect cell or a mammalian cell. Accordingly the promoter region may thus be the native promoter that is associated with the individual insect nuclear hormone receptor gene in nature, provided that the promoter is capable of driving expression in the host cell into which the construct is to be introduced. Alternatively the promoter region may be any one of those with which the skilled man is familiar with for mediating expression in the host of choice. Thus, the promoter region may comprise a constitutive promoter, a tissue- or developmentally-specific promoter, or an inducible promoter. In a particularly preferred embodiment, where the baculovirus expression system and Sf9 or Sf21 cells are to be employed the promoter region will comprise the polyhedrin promoter from the Autographa californica nuclear polyhedrosis virus.

Expression constructs according to this aspect of the invention optionally comprise a transcriptional terminator region. Typically, such a transcriptional terminator region will be one that is suitable for the termination of transcription in the host cell into which the expression construct is to be introduced. Transcriptional terminator regions suitable for this purpose are described in the art.

The present invention also provides a cell transformed with an expression construct as described hereinbefore. Suitable host cells for transformation with a DNA construct of the invention include bacterial cells (such as Escherichia coli), yeast cells (for example, Saccharomyces cerevisiae, Pichia species, Schizosaccharomyces pombe), insect cells (for example, Drosophila S2 cells, Spodoptera frugiperda Sf9 or Sf21 cells, Trichoplusia ni High Five™ cells), and mammalian cells. In a particularly preferred embodiment, an expression construct of the invention is transformed into insect cells, in particular Sf9 cells.

Introduction of the expression construct into a host cell may employ any suitable technique. In eukaryotic cells such techniques include calcium phosphate transfection, DEAE-Dextran, electroporation, particle bombardment, liposome-mediated transfection or transduction using retrovirus, adenovirus or other viruses, such as vaccinia or, for insect cells, baculovirus. In bacterial cells, suitable techniques may include calcium chloride transformation, electroporation or transfection using bacteriophage. In particularly preferred embodiment, Sf9 cells are transformed using baculovirus, using for example, the protocols supplied with the MaxBac®2.0—Complete Baculovirus Expression System (Invitrogen).

The expression construct may also be introduced into a whole organism i.e. an insect, and a stable insect line generated. In one embodiment it is preferred that the expression construct is introduced into Drosophila, using standard transformation techniques (see for example Spradling & Rubin 1982, Science 218:341-347). Thus transgenic Drosophila insects may be produced wherein the level of expression of the insect nuclear hormone receptor gene present in the expression construct is amplified over (i.e. increased above) the level of expression observed in wild-type non-transformed Drosophila.

In yet another aspect of the invention a naturally occurring nuclear hormone receptor in a host insect (or insect cell) may be replaced with a homologue from a different insect species. Typically the host will be a model insect species that is preferably not an agronomic pest-species. Drosophila species are a suitable host species for use in this aspect of the invention. Preferably the homologue that is used to replace the naturally occurring nuclear hormone receptor will be derived from one of the following insect species: Bombyx ori, Tribolium castaneum, Aedes aegyptii, Anopheles sp., Ceratitis capitata, Pectinophora gossypiella, Heliothis virescens, Helicoverpa armigera, Helicoverpa zea, Batrocera dorsalis, Anastrepha suspense, Musca domestica, Stomoxys calcitrans, Lucilia cuprona.

Cells or insects transformed with an expression construct of the invention may be employed in a method of assaying the susceptibility of a compound to detoxification via a pathway regulated by the nuclear hormone receptor gene present in the expression construct. The method comprises: a) culturing in a suitable growth medium a population of cells or an insect transformed with a DNA construct of the invention b) incubating said compound with said population of cells or an insect; and c) determining after said incubation the level of one or more degradation products of said compound in said population of cells or said insect; wherein the presence of a measurable level of one or more degradation products is indicative that said compound is susceptible to degradation by the product of a gene regulated by the nuclear hormone receptor gene present in the expression construct. In particular, the level of said degradation product(s) will be significantly different to the level of the same product(s) in a control insect/insect cell.

The skilled man will readily appreciate that as an alternative to measuring the level of one or more degradation products, the level of the parent test compound may be monitored. If a test compound is susceptible to detoxification or catabolism via metabolic pathway that is regulated by the nuclear hormone receptor (such as for example, DHR96 or a homologue thereof), then the level of parent compound will decrease in non-transgenic insects or insect cells (where the nuclear hormone receptor is expressed). By comparison, in transgenic insects or transgenic insect cells according to the first aspect of the invention, the level of parent compound will be higher, and over time will be maintained at a higher level than in non-transgenic insects or non-transgenic insect cells.

The suitable growth medium will be dependent upon the type of transformed cells of the invention. For example, if the transformed cells of the invention are Saccharomyces cerevisiae yeast cells, YPD is one such suitable medium. Where the transformed cells are Sf9 or Sf21 cells, Grace's Insect Cell Culture Medium is one such medium. Where the transformed cells are Drosophila S2 cells, suitable culture medium is Schneider's insect medium, optionally supplemented with foetal calf serum. The skilled man will be familiar with alternative media and the growth conditions that are suitable for growth of the particular host cells chosen.

Degradation products of the compound being assayed (or presence of the test compound per se) may be measured using any suitable analytical technique. In preferred embodiments LC-MS and/or GC-MS are the techniques employed to detect and measure metabolites and/or test compound. In order to facilitate the measurement of metabolites/test compound, the cells or insects treated with the test compound may be lysed and the cell contents released into a suitable buffer. The skilled man will be familiar with suitable methods for cell lysis which are well-documented in the art.

By comparing the effect of a compound on an insect or insect cell under-expressing an insect nuclear hormone receptor gene with the effect of the same compound on an insect or cell over-expressing the same insect nuclear hormone receptor gene (and optionally also comparing the effect of the compound on a wild type and/or non-transformed insect or insect cell), the susceptibility of that compound to detoxification via a pathway regulated via the encoded nuclear hormone receptor may be assessed. Such a comparison allows better detection of weak insecticidally active compounds that may otherwise not have been identified, since they are susceptible to detoxification by a pathway that is regulated by the insect nuclear hormone receptor. The in vivo metabolism of the compound may then be examined using any suitable analytical technique (for example, an LC-MS technique) and solutions proposed for the enhancement of stability of the compound by altering the chemistry of those parts of the molecule that are subject to degradation. The use of transgenic insects, insect and other cells in this way forms yet another aspect of the invention.

Thus a compound that has been identified or verified as being capable of acting as an insecticide using a transgenic insect or insect cell as described in the first aspect of the invention, may also be tested as described above to assess whether it is susceptible to detoxification via a pathway that is regulated via the nuclear hormone receptor gene whose expression level was reduced in the transgenic insect or insect cell against which the compound has been tested. It will be appreciated by the skilled man that where a compound is tested in this manner, the same nuclear hormone receptor gene will be used in each assay. It will also be appreciated that the assay for the susceptibility to detoxification may be performed initially, and the assay using a transgenic insect or insect cell as described in the first aspect of the invention may be carried out subsequently. Where this is the order in which the assays are carried out, the presence of degradation products in the first assay should correlate with the observation of insecticidal activity in the second assay.

Where it is found that a compound being assayed is susceptible to metabolic detoxification, the assay (or assays) may be used in an iterative manner to aid the design of compounds that are less susceptible to detoxification. For example, if a potential insecticide comprises a backbone chemical structure to which one or more sub-groups are attached, it may be found that by substituting one or more of such sub-groups for a different sub-group and re-testing the modified compound, the modified compound may be found to have an improved resistance to detoxification. If this is the case, the process may be repeated either altering the same, or a different, sub-group and then re-testing the compound in the assay(s) of the invention. In this way compounds may be modified to produce insecticides that are resistant to detoxification whichever pathway(s) is(are) being regulated via the under- or over-expressed insect nuclear hormone receptor.

The skilled man will also appreciate that by comparing the effect of a compound on an insect or insect cell under-expressing an insect nuclear hormone receptor gene with the effect of the same compound on an insect or insect cell over-expressing the same insect nuclear hormone receptor gene (and optionally also comparing the effect of the compound on a wild type and/or non-transformed insect or insect cell), the ability of that compound to act as a pro-pesticide may be assessed. In transgenic insects or transgenic insect cells which over-express the nuclear hormone receptor, it is thought that those metabolic pathways under the control of the nuclear hormone receptor will be up-regulated. Consequently in such transgenic insects and transgenic insect cells, the rate of conversion of pro-insecticide to insecticide is likely to be increase. Thus, transgenic insects or transgenic insect cells that over-express for example DHR96 or a homologue thereof, may be more susceptible to a pro-insecticidal compound than non-transgenic insects or non-transgenic insect cells, and even more susceptible to the pro-insecticidal compound than transgenic insects or transgenic insect cells wherein the level of DHR96 (or homologue thereof) is reduced in comparison to non-transgenic controls.

In yet a further aspect of the invention there is provided a method of identifying or verifying the ability of a compound to act as an insecticide, which comprises: a) culturing in a suitable growth medium a population of insect cells transformed with an expression construct of the invention; b) incubating said compound with said population of cells; and c) determining after said incubation the viability of said cells; wherein a lack of viability of said cells is indicative that said compound is capable of acing as an insecticide. This aspect is particularly useful for identifying or verifying the ability of a compound to act as an insecticide wherein said compound is not susceptible to detoxification mediated by a metabolic pathway that is regulated by the nuclear hormone receptor gene (e.g. dhr96 or a homologue thereof) that is expressed.

A lack of viability of cells is preferably indicated by cell death. However, the slow growth of cells treated with the compound in comparison to cells that have not been treated with the compound, or some other clear phenotypic change in treated cells in comparison to untreated cells, may also be used as a measure of cell viability.

A still further aspect of the invention is a method of identifying a compound that regulates expression of an insect nuclear hormone receptor gene, which comprises: (i) culturing in a suitable growth medium a population of cells transformed with a DNA construct comprising a reporter gene operably linked to the promoter region of the nuclear hormone receptor gene; (ii) incubating said compound with a population of cells according to step (i); (iii) determining the level of expression of said reporter gene in cells that have been incubated with said compound; and (iv) determining the level of expression of said reporter gene in a population of cells according to step (i); wherein, a difference in the levels of reporter gene expression determined at (iii) and (iv) is indicative that said compound is capable of acting as a regulator of expression of said nuclear hormone receptor gene.

DNA constructs for use in this aspect of the invention are referred to herein as “reporter gene constructs” and will comprise any suitable reporter gene available in the art, for example the reporter gene may be a gene encoding : β-galactosidase, luciferase, or a fluorescent protein, such as for example green fluorescent protein or any of the fluorescent proteins described in the Examples. The reporter gene will be operably linked to the promoter region of an insect nuclear hormone receptor gene. Preferably the promoter region will comprise the promoter of the dhr96 gene from D. melanogaster. The promoter regions for use in this aspect of the invention may be isolated and cloned using standard molecular biological techniques.

A yet further aspect of the invention comprises a reporter gene construct wherein the reporter gene is placed under the control of a DHR96 responsive element (or other insect nuclear hormone receptor responsive element), for example a promoter region (e.g. a cyp gene promoter region) that is regulated by DHR96 (or other insect nuclear hormone receptor). In one embodiment such a reporter construct is introduced into a transgenic insect or transgenic insect cell in which DHR96 (or insect nuclear hormone receptor homologue) is over-expressed or heterologously expressed. Such a transgenic insect or insect cell is then used to identify compounds that interact with the DHR96 (or other insect nuclear hormone receptor) ligand binding domain thus causing DHR96 (or other insect nuclear hormone receptor) to interact with the response element driving the reporter gene.

A reporter gene construct for use in this aspect of the invention may be transformed into Drosophila S2 cells using any suitable method available in the art. Where the reporter gene is luciferase the level of reporter gene expression may be assayed using the Luciferase Assay System from Promega.

Compounds that are identified according to the methods of the invention that utilise reporter gene constructs may be useful as additives to existing insecticides that are subject to detoxification via a pathway that is regulated by DHR96, or they may find utility as insecticides in their own right.

For the avoidance of doubt, where a compound is to be “identified” as having the ability to act as an insecticide or to act as a regulator of the expression (or activity) of an insect nuclear hormone receptor gene, that compound has not previously been shown to act as an insecticide or to act as a regulator of expression (or activity) of an insect nuclear hormone receptor gene. Thus any compound that has previously been shown to act in such a way is excluded from the scope of any method of “identifying” a compound.

Where a compound is to be “verified” as having the ability to act as an insecticide, the compound will already have been shown to have insecticidal activity (although such activity may be weak) through some other means.

The present invention also provides a method of assessing the ability of a compound to act as a regulator of the function of DHR96 or its homologues (i.e. a method of identifying regulators of DHR96 or homologue function). The method relies on the induction of expression of a reporter gene, preferably a fluorescent reporter gene and more preferably a fluorescent reporter gene as described herein in the Examples, in response to ligand binding to DHR96 (or to the homologue thereof). The method utilises a chimeric nuclear hormone receptor cassette comprising ligand binding domain (and optionally the hinge region and variable C-terminal F domain) of DHR96 (or its homologue), fused to heterologous eukaryotic DNA binding (DBD) and optionally also to transcription activation (TA) domains. These latter DBD and TA domains can both be derived from, for example, the S. cerevisiae Gal4 protein, or the DBD and the TA domain may be derived from different sources, e.g. the DBD may be derived from the S. cerevisiae Gal4 protein and the TA domain may be derived from the Herpes simplex virus VP16 protein (Sadowski et al. (1988) Nature 335:563-564). In some instances and as described herein for construct 1, the DBD is derived from the S. cerevisiae Gal4 protein. As shown in the Examples, this may be fused to the DHR96 hinge region and the ligand binding domain and the variable C-terminal F domain to create a functional chimeric receptor. Chimeric nuclear hormone receptor cassettes as described above form yet another aspect of the invention.

In conjunction with the chimeric nuclear hormone receptor cassette, the method uses a reporter transgene cassette comprising a reporter gene linked to a cis-regulatory element that is recognized by the DBD in the chimeric nuclear hormone receptor. As described above, preferably the reporter gene encodes a fluorescent protein, in particular any one of the fluorescent proteins whose amino acid sequences are given in the Examples. In a particularly preferred embodiment, where the DBD is the GAL4 DBD, the cis-regulatory element will be an element that is responsive to the GAL4 DBD, for example the Gal4 UAS region. More than one copy of the UAS region may be incorporated in the reporter transgene cassette in order to provide multiple binding sites for the chimeric nuclear hormone receptor to bind to.

Both the chimeric nuclear hormone receptor cassette and the reporter transgene cassette are introduced into either a whole insect or an insect cell. The resulting transgenic insects or transgenic insect cells, which also form a further aspect of the invention, may then be used to identify compounds that bind to the ligand-binding domain of DHR96. Such compounds elicit a conformational change in the chimeric receptor, which permits the heterologous DBD to bind to the cis-regulatory element that controls expression of the reporter gene, and thus cause reporter gene expression. In this way, agonists of DHR96 (or its homologue's) function may be identified. Such agonists may, be co-administered with a compound and potentiate its activity on the target pest. In addition, such chimeric receptors may be used to identify naturally occurring activators of the nuclear hormone receptor activity, and this will be useful in screening for and/or the design of compounds that block activation of the nuclear hormone receptor.

In yet a further aspect of the invention there is provided an antibody against an insect nuclear hormone receptor, in particular against DHR96 of D. melanogaster. Such an antibody may polyclonal or monoclonal and produced using standard immunological techniques. Antibodies of the invention are useful in verifying whether a particular insect nuclear hormone receptor is expressed in a transgenic insect or insect cell of the invention, and also in quantifying the level of such expression.

Various aspects and embodiments of the present invention will now be illustrated in more detail by way of example. It will be appreciated that modification of detail may be made with out departing from the scope of the invention.

### BRIEF DESCRIPTION OF THE FIGURES

FIG. 1 Shows a schematic representation of DNA construct “pBUGIR[dhr96_hairpin]”

FIG. 2 Schematic representation of [A] the chimeric DHR96 receptor construct (construct 1) and [B] the Gal4-responsive amfp486 reporter gene construct (construct 2)

### EXAMPLES

### Example 1

### Construction of Vectors for Use in the Production of Insects Wherein the Level of Expression of an Insect Nuclear Hormone Receptor Gene Is Reduced Relative to Wild Type Insects

Constructs are generated that encode inverted repeats of dhr96 gene fragments, which on expression fold to form a RNA duplex or as referred to herein a hairpin (hp) RNA molecule, which is capable of mediating a RNA interference (RNAi) effect. The constructs generated also include a spacer sequence, which serves to facilitate cloning, between the inverted repeats. The expression of the inverted repeats is under control of an inducible promoter system.

The constructs are generated using site-specific recombinase mediated cloning (1.1). To provide Drosophila DNA template for the cloning, genomic DNA from a wild-type lab strain, Canton-S (Bloomington stock centre, stock number 1; http:/flystocks.bio.Indiana.edu, Drosophila Stock Center, Department of Biology, Indiana University, 1001 E. 3^{rd} St., Bloomington, Ind. 47405-3700, USA) is isolated using standard molecular biology techniques (Sambrook, et al. 1989, Molecular Cloning, A Laboratory Manual, 2^{nd} Edition, Cold Spring Harbor Laboratory, New York; Bender et al. 1983 J. Mol. Biol. 168: 17-33). This is then used as the DNA template for PCR amplification in various steps as described below. The mRNA sequence of dhr96 (EMBL database, accession No: DM36792) that is used for the design of hairpin constructs is shown below as SEQ ID NO 1. The dhr96 coding sequence is marked with the start codon and termination codon in bold and underlined. Bases 58-661 of this sequence (shown in italics and in capital letters) are used to generate the hairpin molecule.

| SEQ ID NO 1: | |
|---|---|
| gttcattaaa atatgtggtg ataacgcgag ctgccgaatc tgcgtgcaat tcgtgcgtTT | 60 |
| GACGTGGGTA CTAACTGCTA TGCTGTCGCG CGGACAGTTG TTCTGATACG CAGAGTTCCT | 120 |
| GCCTCACCAC ACACGACCAC CTCCATTAAA ACCAGCCACC CCCCCCAGCG CCTCCTCCAC | 180 |
| CGACAGCAGC TGCTCCACCG CACCACCAGG AGAGGGGCAA TTAAAAAATC AATCAGAGGG | 240 |
| CCCTAATTGA AAGCTGCCAC CGTCGAA [symbol] TCGCCGCCGA AGAACTGCGC GGTGTGCGGG | 300 |
| GACAAGGCTC TGGGCTACAA CTTCAATGCG GTCACCTGCG AGAGCTGCAA GGCGTTCTTC | 360 |
| CGACGGAACG CGCTGGCCAA GAAGCAGTTC ACCTGCCCCT TCAACCAAAA CTGCGACATC | 420 |
| ACTGTGGTCA CTCGACGCTT CTGCCAGAAA TGCCGCCTGC GCAAGTGCCT GGATATCGGG | 480 |
| ATGAAGAGTG AAAACATTAT GTCCGAGGAG GACAAGCTGA TCAAGCGGCG CAAGATCGAG | 540 |
| ACCAACCGGG CCAAGCGACG CCTCATGGAG AACGGCACGG ATGCGTGCGA CGCCGATGGC | 600 |
| GGCGAGGAAA GGGATCACAA AGCGCCGGCG GATAGCAGCA GCAGCAACCT TGACCACTAC | 660 |
| Tcggggtcac aggactcgca gagctgcggc tcggcggaca gcggggccaa tgggtgctcc | 720 |
| ggcagacagg ccagttcgcc gggcacacag gtcaatccgc ttcagatgac ggccgagaag | 780 |
| atagtcgacc agatcgtatc cgacccggat cgagcctcgc aggccatcaa ccggttgatg | 840 |
| cgcacgcaga aagaggctat atcggtgatg gagaaggtaa tcagctcaca aaaggacgcc | 900 |
| ttaaggctgg tgtcgcattt gatcgactat ccaggcgacg cactcaagat catttcaaag | 960 |
| tttatgaact cgccctttaa cgcgctgaca gtattcacca aattcatgag ctcacccacg | 1020 |
| gacggcgttg aaattatctc aaagatagtt gattcgcccg cggacgtggt ggagttcatg | 1080 |
| cagaacttga tgcactcgcc agaggacgcc atcgatataa tgaacaagtt catgaatacc | 1140 |
| ccagcggagg cgctgcgcat tcttaaccga atcctaagcg gcggaggagc gaacgcagcc | 1200 |
| cagcagacag cagaccgcaa gccattgctg gacaaggagc cggcggtgaa gcctgcagcg | 1260 |
| ccagcggagc gagctgatac tgtcattcaa agcatgctgg gcaacagtcc gccaatttcg | 1320 |
| ccacatgatg ctgccgtgga tctgcagtac cactcgcccg gtgtcgggga gcagcccagt | 1380 |
| acatcgagta gccacccctt gccttacata gccaactcgc cggacttcga tctgaagacc | 1440 |
| ttcatgcaga ccaactacaa cgacgagccc agtctggaca gtgattttag cattaactca | 1500 |
| atcgaatcgg tgctatccga ggtgatccgc attgagtacc aggccttcaa tagcatacaa | 1560 |
| caagcggcat cgcgcgtaaa ggaggagatg tcctacggca ctcagtctac gtacggtgga | 1620 |
| tgcaattcgg ctgcaaacaa tagccagccg cacctgcagc aacccatctg cgccccatcc | 1680 |
| acccagcagt tggatcgcga gctaaacgag gcggagcaaa tgaagctgcg ggagctgcga | 1740 |
| ctggccagcg aggctcttta tgatcccgtg gacgaggacc tcagcgccct gatgatgggc | 1800 |
| gatgatcgca ttaagcccga cgacactcgc cacaacccaa agctattgca gctgatcaat | 1860 |
| ctgacggcgg tggccatcaa gcggcttatc aaaatggcca agaagattac agcattccgt | 1920 |
| gacatgtgcc aggaggacca ggtggcccta ctcaaaggtg gctgcacaga aatgatgata | 1980 |
| atgcgctccg taatgattta cgacgacgat cgcgccgcct ggaaggtacc ccataccaaa | 2040 |
| gagaacatgg gcaacatacg cactgacctg ctcaagtttg ccgaaggcaa tatctacgag | 2100 |
| gagcaccaaa agttcatcac aacgtttgac gagaagtggc gcatggacga gaacataatc | 2160 |
| ctgatcatgt gtgccattgt cctttttacc tcggctcgat cgcgagtgat acacaaagac | 2220 |
| gtgattagat tggaacagaa ttcctactat tatcttctgc gaagatatct ggagagtgtt | 2280 |
| tattctggct gtgaggcgag aaacgcgttt atcaagctaa tccaaaagat ttcagatgtg | 2340 |
| gagcgtctga acaagttcat aattaatgtc tatttgaatg ttaacccatc ccaggtggag | 2400 |
| cccttgctgc gtgaaatatt cgatttgaaa aatcactaga caaccgatgc gtgtcgggca | 2460 |
| tttaatgcct atgttgatgc ccaatgatga atggtcaaca agctgtagtt gttgttgttg | 2520 |
| ttgatgtctg ttttatcttg tcgcttgtaa tgttagattt taatcgaatg tgattgttag | 2580 |
| atttgcatat actgcataga ttttatattt ctacatcaaa gagagcatat ttaggatacc | 2640 |
| aagtgcaaag caacacaatc tatatgtaat gtacaccgtt tacctagttt caaataaact | 2700 |
| agacgataat gcaataacta acttggaagc gtgggttctg tgcaaaaagg aaaaaagaca | 2760 |
| aaaaaaataa actgactttg agaaccagtg gtaaacc | 2797 |

### 1.1 Site-Specific Recombinase Mediated Cloning

The strategy employed in the production of hairpin constructs utilises site-specific phage recombinase mediated cloning (Gateway technology, Invitrogen). The pB-UGIR w+ vector (Genbank AY196824) is used as it has been engineered to contain recombinase target sites that allow recombination of inverted repeats into the vector.

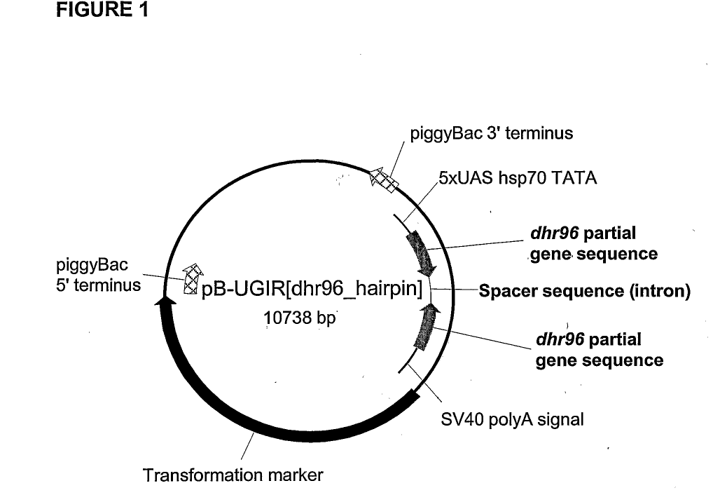

The insert sequence for dhr96 (bases 58-661 of SEQ ID NO 1) is PCR amplified from D. melanogaster genomic DNA using primers dhr96-F (SEQ ID NO 2: 5′ tattgcggatccttgacgtgggtactaactgctatg 3′) and dhr96-R (SEQ ID NO 3: 5′ agtccggaattcagtagtggtcaaggttgctgct 3′) containing the appropriate restriction enzyme target site sequences which facilitate restriction-enzyme-mediated cloning into ENTRY vectors (Invitrogen), such as pENTR2B vector (Invitrogen). Then, in a single step, these ENTRY-derived plasmids are mixed with the pB-UGIR w+ vector, and the desired constructs are generated via recombinase-mediated cloning following the manufacturer's protocols. Using the pB-UGIR w+ vector, the dhr96 sequence inserts are cloned in the tail-to-tail orientation (i.e. the first insert was cloned 5′ to 3′ with the second being cloned 3′ to 5′, see FIG. 1).

### Example 2

### Transformation of D. melanogaster and Generation of Transgenic Flies

Drosophila melanogaster w¹¹¹⁸ embryos (Bloomington stock centre, stock No. 6326) are transformed according to Spradling, A. C. and Rubin, G. M (1982, Science 218:341-347). Briefly, the DNA plasmids for transformation are prepared using a Qiagen Midi kit. Each transformation construct is then co-precipitated and resuspended into injection buffer (5 mM KCl, 0.1 mM NaH₂PO₄, pH 6.8) at a concentration of 0.5-1 μg/μl, together with the piggyBac helper plasmid (pBlu-uTp, Genbank AY196821) at a concentration of 0.1 μg/μl. Embryos are injected prior to pole cell formation; thus 0-30 min old w¹¹¹⁸ embryos are collected, dechorionated and dessicated, and then lined up on glass slides covered with double-sided sticky tape to hold the embryos in place. The embryos are covered with a minimal amount of halocarbon oil, and microinjected posteriorly with the transformation construct and helper plasmid mix. The injected embryos are then incubated at 25° C., and any hatching larvae are recovered and transferred to standard diet. Adults emerging from these larvae are then crossed to w¹¹¹⁸ adults, and stable transformants are selected from their progeny (expressing the mini−w+ marker for transformation). Several independent transgenic flies per injected construct are isolated, and stocks of these transgenic flies are established. Transgenics are also confirmed by PCR amplification of sequences specific to the transformation plasmids.

The transgenic flies thus generated are of the genotypew¹¹¹⁸, pBac{pB-UGIR [dhr96_hairpin]} and the transgene insertions onto any of the chromosomes are balanced if required using standard genetic techniques and the common Balancer chromosomes (Bloomington stock centre; http:/flystocks.bio.Indiana.edu).

### Example 3

### Growth and Maintenance of D. melanogaster Flies

Drosophila melanogaster flies are cultured according to standard methods (as described in http://flystocks.bio.indiana.edu/culturing.htm). The flies are fed on a diet comprising: 100 g agar, 350 g yeast, 300 g treacle, 150 g sucrose, 300 g dextrose, 150 g maizemeal, 100 g wheatgerm, 200 g soya flour mixed in a total of 10 litres of water. This diet mixture is heated to ensure thorough mixing and melting of the agar. After allowing the diet to cool to below 60° C., 10 g nipagen (4-hydroxybenzoic acid methylester) in 10 ml ethanol and 50 ml of proprionic acid is mixed into the diet. The diet is dispensed into culture bottles or vials, and allowed to set before use. The flies are routinely cultured in this diet, and maintained in a 70%-75% humidified 18° C. or 25° C. incubator.

### Example 4

### Induction of Expression of the dhr96 Hairpin RNA Molecule Using a Binary Component System

The transgenic flies generated in Example 2 comprise a dhr96-specific hairpin RNA molecule, and the expression of this hairpin molecule is under the control of an inducible promoter (the UAS promoter). Induction of hairpin expression mediates the degradation of the corresponding endogenous mRNAs through a RNA interference (RNAi) mechanism. As expression of the hairpin molecules is under control of an inducible promoter, an inducing agent is provided to effect the RNAi in these flies. This inducing agent is provided from another transgenic line by means of a genetic cross that brings together the inducing agent and the inducible transgene (i.e. in this case the hairpin molecule) into the same individual flies.

In this type of binary component system, the yeast GAL4 protein acts as a transcriptional regulator and thus as an inducing agent; it binds to UAS promoter sequences and activates the expression of any sequences encoded downstream of this promoter, thus rendering the expression of such sequences inducible by the GAL4 protein (Brand, A. H & Perrimon, N 1993, Development 118:401-415).

This binary system is applied by a genetic cross, between (a) a transgenic fly strain that encodes for the transcription factor (GAL4), and (b) a transgenic fly line that contains the UAS promoter encoded upstream of the cloned dhr96 inverted repeat sequences (generated as described in Example 2). A proportion of the progeny from such a cross will express both the GAL4 protein as well as the hairpin molecule (via GAL4 regulation), and thus express double-stranded (ds) RNA corresponding to the partial dhr96 sequences encoded by the hairpin. These dsRNA molecules mediate degradation of the endogenous mRNA transcripts for dhr96, via an RNAi mechanism. The result is that the amount of endogenous mRNA transcripts for dhr96 is depleted. This effects a reduced level of expression of the dhr96 gene and the product thereof, and a knockdown or knockout of dhr96 gene function is thus observed.

The GAL4-expressing transgenic fly lines used herein are obtained from the Bloomington stock centre (supra) and are described by the genotype: y[I] w[*]; P{w[+mC]=Act5C-GAL4}25FO1/CyO, y[+] (Bloomington stock number 4414); w[*]; P{w[+mW.hs]=GAL4-arm.S}11 (Bloomington stock number 1560); y[1] w[*]; Pt{w[+mC]=Act5C-GAL4}17bFO1/TM6B, Tb[1] (Bloomington stock number 3954). The transgenic fly lines comprising dhr96 inverted repeat region are separately crossed with the GAL-4-expressing transgenic fly lines. At least three independent transgenic lines encoding dhr96 hairpin molecules are used in independent genetic crosses to ensure that the effects seen are not due to position effects of the transgene insertion site. The resulting F1 progeny are selected for those that carry both the GAL4 and the dhr96_hairpin transgene.

The F1 flies from some of these crosses had phenotypes which were attributed to the knockdown of dhr96 expression. For example, the selected F1 progeny from a cross between the strongest allele of genotype w¹¹¹⁸, pBac{pB-UGIR [dhr96_hairpin]} and the driver line (y[1] w[*]*; P{w[+mC]=Act5C-GAL4}25FO1/CyO, y[+] (Bloomington stock number 4414)) showed larval (L3) lethality. Similarly the selected Fl progeny from a cross between a weaker allele of genotype w¹¹¹⁸, pBac{pB-UGIR [dhr96_hairpin]} and the same GAL4 driver as above showed pupal lethality.

The relative dhr96 mRNA depletion in these F1 flies is measured using a Quantitative reverse transcription-polymerase chain reaction (Q-RT-PCR), and compared to wild-type or other suitable control flies. Q-RT-PCR amplification of mRNA is carried out on pools of the F1 flies (between ten and twenty, 0-3 day old, adults were used). The reference endogenous RNA sample used for relative quantitation is RpL32. The primers and Taqman probes used for this mRNA detection assay are (sequences given in the 5′ to 3′ direction):

| | | |
|---|---|---|
| Dhr96 Q-RT-PCR assay | | |
| dhr96-fwd_primer | (SEQ ID NO 4) | CATGGACGAGAACATAATCCTGAT |
| dhr96-rev_primer | (SEQ ID NO 5) | CAGAAGATAATAGTAGGAATTCTGTTCCAA |
| dhr96-taqman_probe | (SEQ ID NO 6) | TGTGCCATTGTCCTTTTTACCTCGGCT |
| Rpl32 Q-RT-PCR assay | | |
| RpL32-fwd_primer | (SEQ ID NO 7) | GATATGCTAAGCTGTCGCACAAAT |
| RpL32-rev_primer | (SEQ ID NO 8) | GGCATCAGATACTGTCCCTTGAA |
| RpL32-taqman_probe | (SEQ ID NO 9) | CGCAAGCCCAAGGGTATCGACAAC |

For the F1 progeny that were generated as described above, the assays showed that overall dhr96 expression was reduced in comparison to the control flies.

### Example 5

### Generation of Anti-DHR96 Polyclonal Antibodies

Antibodies are raised to a peptide from the DHR96 protein. This peptide is selected (a) to maximise the probability that antibodies generated against—it are specific to DHR96, and (b) from regions predicted to have good antigenicity (using Windows 32 Protean 5.03 software, DNASTAR Inc.). The nucleotide sequences encoding the peptide fragment from DHR96 (-DHR96_peptide2 SEQ ID NO 10) are cloned and expressed, using standard molecular biology techniques (Sambrook, et al. 1989, Molecular Cloning, A Laboratory Manual, 2^{nd} Edition, Cold Spring Harbor Laboratory, New York). The peptide is tagged at the 5′ end with maltose-binding protein (MBP) to facilitate later purification steps.

DHR96_peptide2 (SEQ ID NO 10; amino acids-251-513
from SWISSPROT accession No Q24143):
TDGVEIISKIVDSPADVVEFMQNLMHSPEDAIDIMNKFMNTPAEALRILN
RILSGGGANAAQQTADRKPLLDKEPAVKPAAPAERADTVIQSMLGNSPPI
SPHDAAVDLQYHSPGVGEQPSTSSSHPLPYIANSPDFDLKTFMQTNYNDE
PSLDSDFSINSIESVLSEVIRIEYQAFNSIQQAASRVKEEMSYGTQSTYG
GCNSAANNSQPHLQQPICAPSTQQLDRELNEAEQMKLRELRLASEALYDP
VDEDLSALMMGDD

The MBP-DHR96-fusion peptide is expressed, the MBP cleaved off, and the DHR96 peptide purified. Two New Zealand white rabbits are immunised with this DHR96 peptide immunogen. The immunoglobulin fractions are purified by affinity chromatography on Protein A-Sepharose. Additionally, any anti-MBP antibodies are removed from the immunoglobulin fractions by affinity chromatography on MBP-Sepharose. The antibodies are assessed for reactivity towards the immunising peptide by immunoblotting against purified DHR96 peptide.
(1) Non-specific binding is checked for using pre-immune sera from the two rabbits. The purified peptide is loaded onto an SDS-PAGE gel at amounts of 1, 5 and 10 ng per well. Pre-immune sera at concentrations of 1/5000 showed no cross-reactivity to the peptide.
(2) Specific binding is tested for using the immune sera recovered from the two rabbits. The purified peptide is loaded onto an SDS-PAGE gel at amounts of 1, 5 and 10 ng per well. Immune sera at concentrations of 1/5000 showed specific binding most prominently to the peptide at ˜29 kDa as expected. Some background binding was also observed, the sizes of which matched the additional bands seen in the purified peptide sample itself.

Polyclonal antibodies against Drosophila melanogaster DHR96 were thus generated. Levels of DHR96 protein in Drosophila melanogaster can be investigated with these antibodies using standard methodology for sample extraction and Western blotting.

### Example 6

### Modified Response to Xenobiotics in dhr96 Knockdown Insect Strains 6.1 Bioassays

Bioassays are set up as follows to expose the insects to test compounds: for testing on adults by contact and feeding, a 5% sucrose: 1% agar mix is prepared and 0.75 mLs poured into each well of a 24-well plate, and allowed to set. The test compound is dissolved in a suitable ethanol-acetone based solvent system to the dilutions required. 20 μL of a single dilution is spread uniformly on the surface of the sucrose-agar base in a well. By applying the dilution series to consecutive wells in a plate, a dose range of the compound is created. The control wells contain formulation solution only, applied similarly to the surface of the sucrose-agar base.

The test flies used in such a bioassay are described in Example 4, namely F1 progeny selected to carry both the GAL4 and the dhr96_hairpin transgene. In these flies levels of the dhr96 mRNA is reduced. Suitable control fly strains are also used.

10 (0-3 day old) adult flies of the appropriate genotypes as described above are added to each well, after the applied compound solution has dried. The plates are sealed with a breatheable film, and incubated under normal culture conditions (25° C., 70-75% humidity). Three replicates per genotype and per dose are included, and the experiment is ideally repeated on 3 separate ocassions.

For testing on larvae by contact and feeding, the test compound is dissolved in a suitable ethanol-acetone based solvent system and mixed in well with normal fly diet to the dilutions required. 1.5 mLs of the diet is poured into each well of a 24-well plate, and allowed to set. By applying the dilution series to consecutive wells in a plate, a dose range of the compound is created. The control wells contain formulation solution only, mixed in similarly with fly diet.

The eggs used to infest the wells in this larval bioassay are obtained as described above, and consist of F1 progeny selected to carry both the GAL4 and the dhr96_hairpin transgene. In these insects the levels of the dhr96 mRNA is reduced. Suitable control fly strains are also used.

25 eggs of this genotype are added to each well, after the diet has set. The plates may be sealed with a breathable film, and incubated under normal culture conditions (25° C., 70-75% humidity). Three replicates per genotype and per dose are included, and the experiment is ideally repeated on 3 separate ocassions.

For testing on adults by topical application of compounds, a method has been adapted from Dapkus (Drosophila Information Newsletter, Volume 1, January 1991; http://jfly.iam.u-tokyo.ac.jp/html/din/DIN_Vol_01_{—}9101.txt). 0-3 day old adult D. melanogaster females are anaesthetised using CO₂, and 0.4 uL of compound dissolved in acetone is applied to the abdomens of these females using a Hamilton pipette. After compound application, the flies are cultured under normal conditions for the required time intervals, and either visually assessed (as in Example 6.2) or harvested for chemical analysis (Example 7). Ideally the experiment is repeated on 3 separate ocassions

### 6.2 Assessment

The effects of the compounds on the whole insects (larvae and adults) are monitored by visual assessment, daily or at a frequency as required. In one type of assessment, the mortality of the insects over time is scored. For example, if the adult test is used, the insects are monitored at time points that have been determined previously to be suitable for the experiment. The number of dead flies at each time point is scored.

In addition, for the bioassay where compounds are applied topically, the flies may also be harvested for chemical analysis. At the time for collection, the flies are immediately frozen and stored at −80° C. They are then processed and analysed as described in Example 7.

### 6.2.1 Results

Bioassays are carried out as described above. In one example, the insecticide Malathion was applied to 20 females at a dose of 50 ppm per time point to be assessed, according to the topical application method described above. The time points at which the flies were collected were 0, 1, 2 and 5 hours after compound application. The strains tested were:
CONTROL 1:w[*]; P{w[+mW.hs]=GAL4-arm.S}11 (Bloomington stock number 1560)
CONTROL 2:w¹¹¹⁸; pBac{pB-UGIR [dhr96_hairpin]}
MUTANT (generated as described in Example 4 above): w[*];,pBac{pB-UGIR [dhr96_hairpin]}/P{w[+mW.hs]=GAL4-arm.S}11
   In 3 independant experiments a trend was observed: whilst the control strains show an increasing percentage mortality over time (to ≧95% mortality by 5 hours), the mutant strain showed either no or significantly reduced mortality at 5 hours. This result is consistent with the requirement for malathion to be converted from a propesticide to a pesticide by the action of metabolic enzymes. One explanation for this is that the metabolic enzymes required for this conversion are not induced in the mutant strains due to reduced expression of dhr96.

### Example 7

### Modified Rate of (Insecticidal) Compound Degradation in dhr96 Knockdown Insect Strains

### 7.1 Bioassays

Bioasays for topical application are carried out as described in Example 6.1. Test compounds include: DDT (1,1,1-trichloro-2,2-bis(4-chlorophenyl)ethane) at 100 ppm; Imidacloprid at 5 ppm; Malathion at 50 ppm and Carbaryl at 100 ppm.

The D. melanogaster strains used in this experiment include:
CONTROL 1:w[*]; P{w[+mW·hs]=GAL4-arm·S}11 (Bloomington stock number 1560)
CONTROL 2: w¹¹¹⁸;pBac{pB-UGIR [dhr96_hairpin]}
MUTANT (generated as described in Example 4 above): w[*];pBac{pB-UGIR [dhr96_hairpin]}/P{w[+m W·hs]=GAL4-arm·S}11

Between 20-40 adult flies per strain and time point are used, depending on the compound being tested. The time points at which the insects are collected and frozen are typically 0, 1, 2, 5, 8 hours after compound application.

7.2 Chemical analysis

The samples collected and frozen at the time points described above are extracted twice in acetonitrile and reconstituted in acetonitrile, as described below. Final quantitative determination of parent compound and/or any metabolites is by high performance Liquid Chromatography coupled to Mass Spectrometry (LC-MS) or Gas Chromatography Mass Spectrometry (GC-MS), whichever technique is most appropriate for the compound under study.

### 7.2.1 Sample Preparation and Extraction

Sample extraction is carried out using Fast Prep Lysing Matrix A tubes (Q-Bio-gene; 2 mL tubes, Part No. 6910-100, Anachem Ltd) and a FastPrep™ machine (FastPrep™ FP120 Bio 101 Savant sample shaker, Anachem Ltd ).

### Extraction:

a. The collected insect samples are homogenised in Fast Prep Lysing Matrix A tubes for 10 seconds while still frozen using the FastPrep™ shaker, until the samples are powdery homogenates.
b. 0.6 mL of acetonitrile is added and the samples shaken for a 20 sec burst in the FastPrep™ shaker. The samples are left to incubate for 5 min and then shaken again for another 20 sec.
c. The samples are centrifuged for 5 min at 13,000 rpm in a standard bench-top centrifuge, and the extract (supernatant) is separated from the solid matrix by quantitatively transferring the supernatant into a graduated clean vial (4 mL) using a Pasteur pipette.
d. The sample extraction with acetonitrile is repeated (0.6 mL) using 2×20 sec bursts and 5 min incubation in between. The samples are again centrifuged as above, and the extract is separated from the solid matrix by transferring the supernatant quantitatively into the vial (4 mL) containing the first extract. Complete transfer of extracts is ensured. The vials are shaken to allow mixing of the two extracts.
e. The extracted samples are centrifuged at 3500 rpm for 5 min in a standard bench-top centrifuge.
f. The full amount of each extracted sample (1.2 mL) is transferred into a 1.5 mL Eppendorf tube and evaporated to dryness using a stream of air at 45° C. The samples are reconstituted in acetonitrile (to 100 μL per 20 flies for DDT analysis). To aid complete re-dissolution of the analytes, the samples are ultra-sonicated (in a standard ultra-sonication bath) for 2×1 min and to ensure that any residue adhered to the tube wall is reconstituted.
g. The samples are then centrifuged at 13000 rpm for 5 min in a standard bench-top centrifuge.
h. The clean supernatant is transferred into a suitable insert vial (250 μL) for LC-MS and/or GC-MS analysis.
   7.2.2 Analysis of Parent Compound and/or Metabolites Using LC-MS and GC-MS

LC-MS analysis: Imidacloprid, malathion and carbaryl, and/or their metabolites, are analysed on a triple-quadrupole mass spectrometer (TSQ Quantum Discovery, Thermo-Electron Corporation) interfaced with a liquid chromatograph (HP1100, Agilent Technologies) and autosampler (HTS-PAL, CTC Analytics). The instrument control and acquisition software are Thermo Xcalibur version 1.3.

Chromatography is performed with a 150×3 mm Phenomenex Synergi Hydro-RP column, particle size 4 μm, pore size 80 Å, using a 10 μl injection volume. The following gradient is applied at a flow rate of 1 ml min⁻¹ with a split of 5:1 (waste:MS) between the HPLC column and the mass spectrometer:

| | | % Acetonitrile + |
|---|---|---|
| Time (min) | % Water + 0.2% formic acid | 0.2% formic acid |
| 0 | 80 | 20 |
| 1 | 80 | 20 |
| 10 | 15 | 85 |
| 12 | 15 | 85 |
| 12.5 | 80 | 20 |
| 16 | 80 | 20 |

For the parent compounds, the mass spectrometer is operated in the single reaction monitoring (SRM) mode using argon as the collision gas at 1.5 mTorr, observing the following transitions with a dwell time of 0.3 s and window of 1 Da:

| | Parent Ion | Fragment ion | Collision Energy/ |
|---|---|---|---|
| Compound | (m/z) | (m/z) | eV |
| Carbaryl | 202 | 145 | 12 |
| Imidacloprid | 256 | 209 | 20 |
| Malathion | 331 | 127 | 15 |

Potential metabolites are detected by examining total ion chromatograms for peaks of possible interest that might be derived from the parent compound on the basis of one or more of a combination of: a comparison to previously analysed standard compound; the molecular weight relative to the parent; the isotope pattern (e.g. in cases where the parent contains isotopes of atoms such as chlorine); and the fragmentation pattern of full-scan spectra derived from peaks in the total ion chromatogram. The relative accumulation and subsequent disappearance of metabolites over time is assessed by integration of the ion peak area.

GC-MS analysis: DDT and/or its metabolites is analysed with an Agilent gas chromatograph HP6890 equipped with a Varian CP-SIL 8 capillary column (30 m×0.25 mm, with 0.25 μm stationary phase film). Samples are injected splitless with the injector temperature 250° C., using an Optic 2 (ATAS) programmed temperature GC inlet. Helium is used as carrier gas at an initial pressure of 9.60 psi and a final pressure of 22.01 psi with electronic pressure control to obtain a 1 mL/min flow rate through the run (27.50 min). The gas chromatograph is directly coupled to an Agilent HP5973 mass spectrometer working in electron impact ionisation mode at 70 eV, and scanned masses in the range of 50-800 Da for full scan data acquisition. The column oven temperature program is as follows: 60° C. (isothermal for 1 min), heating rate to 180° C. at 30° C./min, to 250° C. at 4° C./min, final isothermal temperature of 250° C. (held for 5 min). The transfer line, quadrupole mass filter and ion source temperatures are kept at 280, 150 and 230° C. respectively. Data are acquired in full scan and selected-ion monitoring (SIM) modes and processed with the Agilent Chemstation software.

The parent insecticidal compound (DDT) is identified by comparing retention times and mass spectra of its peak to those of a standard compound analysed previously. Quantitation of DDT is performed in selected-ion monitoring (SIM) mode with the acquisition of a primary (m/z=235) and two confirmation ions (m/z=237 and 165) of the target compound. Quantitation is performed by integration of the primary ion peak area (m/z=235 ion chromatogram). The scan time during data acquisition is set at 1.0 s with three microscans per second.

Peaks corresponding to potential DDT metabolites are similarly identified by comparing their retention times and associated mass spectra peak to either a standard compound analysed previously or a compound fingerprint from a spectral library, Integration of the relevant ion peak area enables assessment of the relative accumulation and/or disappearance of potential metabolites over time.

### 7.3.1 Data Analysis

The analysis described above in 7.1 and 7.2 is carried for the 3 different genotypes listed in 7.1, in three independent (replicate) experiments. The rate of parent compound loss, and/or the rate of metabolite change, is plotted for each genotype over the various time points tested.

### Example 8

### Chimeric Receptors and Reporter Gene Expression (for Detecting a Nuclear-Receptor-Mediated Molecular Response to Chemical Exposure) 8.1 Chimeric Nuclear Receptor Construct Generation

The chimeric nuclear receptor is constructed comprising the hinge region and the ligand binding domain (and optionally the variable C-terminal F domain) of DHR96 (or its homologue), fused to heterologous eukaryotic DNA binding (DBD) and optionally also to transcription activation (TA) domains. These latter DBD and TA domains can both be derived from the S. cerevisiae Gal4 protein, or the DBD can be derived from the S.cerevisiae Gal4 protein and the TA domain can be derived from the Herpes simplex virus VP16 protein (Sadowski et al. (1988) Nature 335:563-564). In some instances and as described for construct 1 below, the DBD is derived from the S. cerevisiae Gal4 protein. This is fused to the DHR96 hinge region and the ligand binding domain and the variable C-terminal F domain to create a functional chimera for this assay.

In the chimeric DHR96 receptor created, the GAL4 DNA-binding domain enables binding of the chimeric DHR96 receptor to a sequence-specific response element in the reporter transgene cassette. The reporter transgene comprises a GAL4-responsive promoter element (UAS) that is upstream of, and regulates expression of, a reporter gene, preferably a fluorescent reporter gene (see Example 8.2).

The expression of the chimeric DHR96 receptor is regulated in a tissue- or temporal-specific manner using any appropriate promoter. Alternatively it may be regulated by means of an external stimulus/signal, for example, when the chimeric DHR96 receptor is ectopically expressed in Drosophila, a heat shock promoter can be used to temporally regulate gene expression (Lis et al. (1983) Cell 35:403; Struhl (1985) Nature 318:677; Schneuwly et al. (1987) Nature 325:816). Using this approach, the level of ectopic gene expression can be easily modulated by altering the temperature and/or duration of the heat treatment.

### Construct 1 (Chimeric DHR96 Receptor Construct) Is Generated as Follows:

The parent vector used for cloning is pB-MCS w+(Genbank accession no. AY196822). Using standard molecular biology methods (Sambrook, et al. 1989, Molecular Cloning, A Laboratory Manual, 2^{nd} Edition, Cold Spring Harbor Laboratory, New York), the selected promoter sequence (e.g. hsp70 promoter sequence), partial dhr96 cDNA sequence and gal4 DBD coding sequence are isolated from genomic DNA or cDNA (wild-type Drosophila melanogaster or S. cerevisiae) as appropriate using PCR amplification.

The hsp70 promoter sequence from Drosophila is used to drive expression of the chimeric DHR96 receptor. Primers are designed to selectively amplify nucleotides 1265-1704 of the Drosophila hsp70 promoter sequence from Genbank accession no. J01104. The isolated promoter sequence is used to drive heat-shock mediated expression of the DHR96 chimeric receptor, using standard heat-shock induction protocols available in the art.

Bases 1265-1704 of the Drosophila hsp70 promoter
sequence from Genbank accession no. J01104
(SEQ ID NO 11):
ATCCCAAAACAAACTGGTTATTGTGGTAGGTCATTTGTTTGGCAGAAAGA
AAACTCGAGAAATTTCTCTGGCCGTTATTCGTTATTCTCTCTTTTCTTTT
TGGGTCTCTCCCTCTCTGCACTAATGCTCTCTCACTCTGTCACACAGTAA
ACGGCATACTGCTCTCGTTGGTTCGAGAGAGCGCGCCTCGAATGTTCGCG
AAAAGAGCGCCGGAGTATAAATAGAGGCGCTTCGTCTACGGAGCGACAAT
TCAATTCAAACAAGCAAAGTGAACACGTCGCTAAGCGAAAGCTAAGCAAA
TAAACAAGCGCAGCTGAACAAGCTAAACAATCTGCAGTAAAGTGCAAGTT
AAAGTGAATCAATTAAAAGTAACCAGCAACCAAGTAAATCAACTGCAACT
ACTGAAATCTGCCAAGAAGTAATTATTGAATACAAGAAGA

Gal4 DNA binding domain sequences are isolated using PCR amplification from S. cerevisiae genomic DNA.

Nucleotides 443-883 (SEQ ID NO 12) from EMBL accession No K01486 comprise the DNA binding domain of the Gal4 protein, and are selectively amplified using PCR with appropriately designed primers.

SEQ ID NO 12:
ATGAAGCTACTGTCTTCTATCGAACAAGCATGCGATATTTGCCGACTTAA
AAAGCTCAAGTGCTCCAAAGAAAAACCGAAGTGCGCCAAGTGTCTGAAGA
ACAACTGGGAGTGTCGCTACTCTCCCAAAACCAAAAGGTCTCCGCTGACT
AGGGCACATCTGACAGAAGTGGAATCAAGGCTAGAAAGACTGGAACAGCT
ATTTCTACTGATTTTTCCTCGAGAAGACCTTGACATGATTTTGAAAATGG
ATTCTTTACAGGATATAAAAGCATTGTTAACAGGATTATTTGTACAAGAT
AATGTGAATAAAGATGCCGTCACAGATAGATTGGCTTCAGTGGAGACTGA
TATGCCTCTAACATTGAGACAGCATAGAATAAGTGCGACATCATCATCGG
AAGAGAGTAGTAACAAAGGTCAAAGACAGTTGACTGTATCG

The above nucleotide sequence encodes amino acids 1-147 (SEQ ID NO 13) of the S. cerevisiae Gal4 protein (i.e. encodes the DBD of the GAL4 protein).

SEQ ID NO 13:
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVS

Selected dhr96 sequences are isolated using PCR amplification from a cDNA library or from wild-type Drosophila genomic DNA as described previously (see Example 1). The sequences to be cloned include the DHR96 hinge region, the ligand binding domain and the F domain, and are encoded by nucleotides 517- 2460 of EMBL:AY051486.

SEQ ID NO 14 (Nucleotides 517-2460 as described
from EMBL:AY051486):
AACATTATGTCCGAGGAGGACAAGCTGATCAAGCGGCGCAAGATCGAGAC
CAACCGGGCCAAGCGACGCCTCATGGAGAACGGCACGGATGCGTGCGACG
CCGATGGCGGCGAGGAAAGGGATCACAAAGCGCCGGCGGATAGCAGCAGC
AGCAACCTTGACCACTACTCGGGGTCACAGGACTCGCAGAGCTGCGGCTC
GGCGGACAGCGGGGCCAATGGGTGCTCCGGCAGACAGGCCAGTTCGCCGG
GCACACAGGTCAATCCGCTTCAGATGACGGCCGAGAAGATAGTCGACCAG
ATCGTATCCGACCCGGATCGAGCCTCGCAGGCCATCAACCGGTTGATGCG
CACGCAGAAAGAGGCTATATCGGTGATGGAGAAGGTAATCAGCTCACAAA
AGGACGCCTTAAGGCTGGTGTCGCATTTGATCGACTATCCAGGCGACGCA
CTCAAGATCATTTCAAAGTTTATGAACTCGCCCTTTAACGCGCTGACAGT
ATTCACCAAATTCATGAGCTCACCCACGGACGGCGTTGAAATTATCTCAA
AGATAGTTGATTCGCCCGCGGACGTGGTGGAGTTCATGCAGAACTTGATG
CACTCGCCAGAGGACGCCATCGATATAATGAACAAGTTCATGAATACCCC
AGCGGAGGCGCTGCGCATTCTTAACCGAATCCTAAGCGGCGGAGGAGCGA
ACGCAGCCCAGCAGACAGCAGACCGCAAGCCATTGCTGGACAAGGAGCCG
GCGGTGAAGCCTGCAGCGCCAGCGGAGCGAGCTGATACTGTCATTCAAAG
CATGCTGGGCAACAGTCCGCCAATTTCGCCACATGATGCTGCCGTGGATC
TGCAGTACCACTCGCCCGGTGTCGGGGAGCAGCCCAGTACATCGAGTAGC
CACCCCTTGCCTTACATAGCCAACTCGCCGGACTTCGATCTGAAGACCTT
CATGCAGACCAACTACAACGACGAGCCCAGTCTGGACAGTGATTTTAGCA
TTAACTCAATCGAATCGGTGCTATCCGAGGTGATCCGCATTGAGTACCAG
GCCTTCAATAGCATACAACAAGCGGCATCGCGCGTAAAGGAGGAGATGTC
CTACGGCACTCAGTCTACGTACGGTGGATGCAATTCGGCTGCAAACAATA
GCCAGCCGCACCTGCAGCAACCCATCTGCGCCCCATCCACCCAGCAGTTG
GATCGCGAGCTAAACGAGGCGGAGCAAATGAAGCTGCGGGAGCTGCGACT
GGCCAGCGAGGCTCTTTATGATCCCGTGGACGAGGACCTCAGCGCCCTGA
TGATGGGCGATGATCGCATTAAGCCCGACGACACTCGCCACAACCCAAAG
CTATTGCAGCTGATCAATCTGACGGCGGTGGCCATCAAGCGGCTTATCAA
AATGGCCAAGAAGATTACAGCATTCCGTGACATGTGCCAGGAGGACCAGG
TGGCCCTACTCAAAGGTGGCTGCACAGAAATGATGATAATGCGCTCCGTA
ATGATTTACGACGACGATCGCGCCGCCTGGAAGGTACCCCATACCAAAGA
GAACATGGGCAACATACGCACTGACCTGCTCAAGTTTGCCGAAGGCAATA
TCTACGAGGAGCACCAAAAGTTCATCACAACGTTTGACGAGAAGTGGCGC
ATGGACGAGAACATAATCCTGATCATGTGTGCCATTGTCCTTTTTACCTC
GGCTCGATCGCGAGTGATACACAAAGACGTGATTAGATTGGAACAGAATT
CCTACTATTATCTTCTGCGAAGATATCTGGAGAGTGTTTATTCTGGCTGT
GAGGCGAGAAACGCGTTTATCAAGCTAATCCAAAAGATTTCAGATGTGGA
GCGTCTGAACAAGTTCATAATTAATGTCTATTTGAATGTTAACCCATCCC
AGGTGGAGCCCTTGCTGCGTGAAATATTCGATTTGAAAAATCAC

The above nucleotide sequence encodes the DHR96 hinge region, the ligand binding domain and the F domain i.e. amino acids 76-723 (SEQ ID NO 15) from SWISSPROT accession No Q24143:

SEQ ID NO 15:
NIMSEEDKLIKRRKIETNRAKRRLMENGTDACDADGGEERDHKAPADSSS
SNLDHYSGSQDSQSCGSADSGANGCSGRQASSPGTQVNPLQMTAEKIVDQ
IVSDPDRASQAINRLMRTQKEAISVMEKVISSQKDALRLVSHLIDYPGDA
LKIISKFMNSPFNALTVFTKFMSSPTDGVEIISKIVDSPADVVEFMQNLM
HSPEDAIDIMNKFMNTPAEALRILNRILSGGGANAAQQTADRKPLLDKEP
AVKPAAPAERADTVIQSMLGNSPPISPHDAAVDLQYHSPGVGEQPSTSSS
HPLPYIANSPDFDLKTFMQTNYNDEPSLDSDFSINSIESVLSEVIRIEYQ
AFNSIQQAASRVKEEMSYGTQSTYGGCNSAANNSQPHLQQPICAPSTQQL
DRELNEAEQMKLRELRLASEALYDPVDEDLSALMMGDDRIKPDDTRHNPK
LLQLINLTAVAIKRLIKMAKKITAFRDMCQEDQVALLKGGCTEMMIMRSV
MIYDDDRAAWKVPHTKENMGNIRTDLLKFAEGNIYEEHQKFITTFDEKWR
MDENIILIMCAIVLFTSARSRVIHKDVIRLEQNSYYYLLRRYLESVYSGC
EARNAFIKLIQKISDVERLNKFIINVYLNVNPSQVEPLLREIFDLKNH

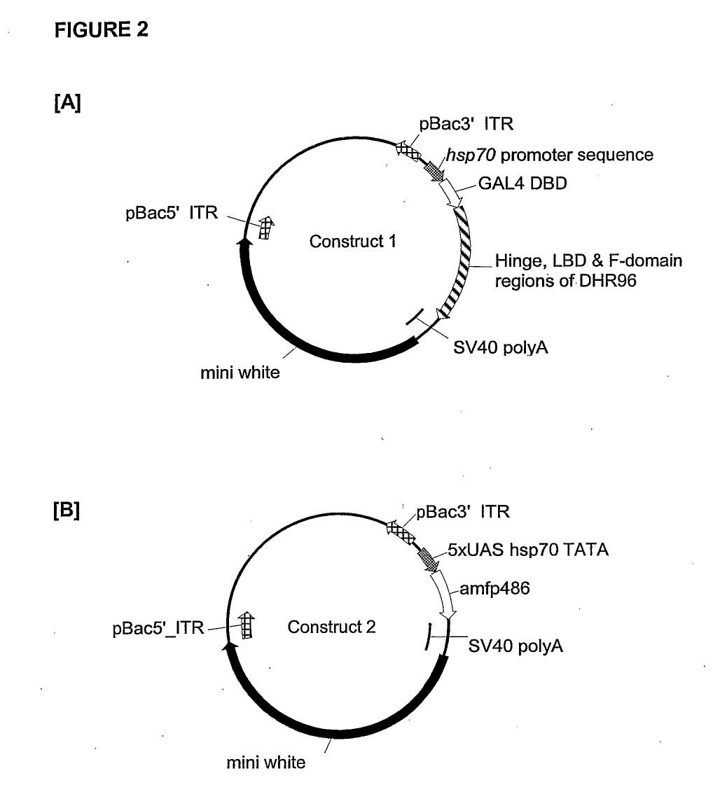

Isolated promoter, dhr96 and gal4 sequences are cloned into the parent vector pB-MCS w+ (Genbank accession no. AY196822) using standard restriction enzyme mediated cloning methods, and a final construct assembled that contains the hsp70 promoter sequence, followed by the GAL4 sequence, followed by the DHR96 fragment as shown in FIG. 2, panel A.

### 8.2 Gal4-Responsive Reporter Construct Generation

The parent vector used for cloning was pB-UAS w+(Genbank accession no. AY196823). Using standard molecular biology methods (Sambrook, et al. 1989, Molecular Cloning, A Laboratory Manual, 2^{nd} Edition, Cold Spring Harbor Laboratory, New York), the selected reporter gene sequence was isolated from genomic DNA or cDNA or other sources sources (such as commercially available plasmids), using PCR amplification or other methods.

The parent vector pB-UAS w+ contains multiple copies (5) of the GAL4-responsive element UAS. These provide multiple sites for binding of the chimeric receptor.

The selected reporter gene sequence is isolated using PCR amplification from an template DNA (Clontech). The reporter gene used encodes the gfp-like fluorescent chromoprotein amfp486 (Swissprot accession No Q9U6Y6), the amino acid sequence of which is given below (SEQ ID NO 16).

SEQ ID NO 16:
MALSNKFIGDDMKMTYHMDGCVNGHYFTVKGEGNGKPYEGTQTSTFKVTM
ANGGPLAFSFDILSTVFKYGNRCFTAYPTSMPDYFKQAFPDGMSYERTFT
YEDGGVATASWEISLKGNCFEHKSTFHGVNFPADGPVMAKKTTGWDPSFE
KMTVCDGILKGDVTAFLMLQGGGNYRCQFHTSYKTKKPVTMPPNHVVEHR
IARTDLDKGGNSVQLTEHAVAHITSVVPF

This reporter gene sequence was cloned into the parent vector using standard restriction enzyme mediated cloning methods, and a final construct (construct 2, FIG. 2 panel B) was assembled that contains the reporter sequence encoded downstream of UAS promoter sequences that are responsive to Gal4.

Other suitable reporter gene sequences which may be similarly isolated using PCR amplification from an appropriate source include those encoding:
a) the gfp-like non-fluorescent chromoprotein fp595 (asfp595), Swissprot accession No Q9GZ28 (the amino acid sequence of which is SEQ ID NO 17).

SEQ ID NO 17:
MASFLKKTMPFKTTIEGTVNGHYFKCTGKGEGNPFEGTQEMKIEVIEGGP
LPFAFHILSTSCMYGSKTFIKYVSGIPDYFKQSFPEGFTWERTTTYEDGG
FLTAHQDTSLDGDCLVYKVKILGNNFPADGPVMQNKAGRWEPATEIVYEV
DGVLRGQSLMALKCPGGRHLTCHLHTTYRSKKPASALKMPGFHFEDHRIE
IMEEVEKGKCYKQYEAAVGRYCDAAPSKLGHN;

b) the gfp-like fluorescent chromoprotein fp538 (zfp538), Swissprot accession No Q9U6Y4 (the amino acid sequence of which is SEQ ID NO 18):

SEQ ID NO 18

MAHSKHGLKEEMTMKYHMEGCVNGHKFVITGEGIGYPFKGKQTINLCVIE
GGPLPFSEDILSAGFKYGDRIFTEYPQDIVDYFKNSCPAGYTWGRSFLFE
DGAVCICNVDITVSVKENCIYHKSIFNGMNFPADGPVMKKMTTNWEASCE
KIMPVPKQGILKGDVSMYLLLKDGGRYRCQFDTVYKAKSVPSKMPEWHFI
QHKLLREDRSDAKNQKWQLTEHAIAFPSALA;

c) the gfp-like fluorescent chromoprotein fp506 (zfp506), Swissprot accession No Q9U6Y5 (the amino acid sequence of which is SEQ ID NO 19):

SEQ ID NO 19

MAQSKHGLTKEMTMKYRMEGCVDGHKFVITGEGIGYPFKGKQAINLCVVE
GGPLPFAEDILSAAFNYGNRVFTEYPQDIVDYFKNSCPAGYTWDRSFLFE
DGAVCICNADITVSVEENCMYHESKFYGVNFPADGPVMKKMTDNWEPSCE
KIIPVPKQGILKGDVSMYLLLKDGGRLRCQFDTVYKAKSVPRKMPDWHFI
QHKLTREDRSDAKNQKWHLTEHAIASGSALP

### 8.3 Transgenic Insect Generation

Transgenic insects that have the two constructs described in Examples 8.1 and 8.2 independently integrated in multiple strains are generated as described above in Examples 2 and 3.

### 8.4 Reporter System Assembly

The transgenic insects generated in Example 8.3 contain either a DHR96-GAL4 chimeric transgene, or a UAS-reporter transgene. When the two transgenes are brought together by means of a genetic cross, the ligand binding domain of the chimeric nuclear receptor can bind a chemical ligand thereby eliciting a conformational change, which then allows the transactivation domain of the chimeric receptor to activate transcription of the reporter gene. The induction of reporter gene expression therefore depends on ligand binding to the chimeric DHR96 receptor.

This system is assembled by a genetic cross between (a) a transgenic fly strain that encodes for the DHR96-GAL4 chimeric protein, and (b) a transgenic fly line that contains the UAS-reporter transgene. A proportion of the F1 progeny from such a cross express the DHR96-GAL4 chimeric protein, and are capable (on ligand binding) of expressing the reporter protein via GAL4 regulation.

At least three independent transgenic lines encoding the transgenes are used in independent genetic crosses to ensure that the effects seen aree not due to position effects of the transgene insertion site.

### 8.5 Bioassay to Detect Chemical Binding to the DHR96 Ligand Binding Domain and Consequent Functional Expression of the Reporter Gene

The effects of compounds on the chimeric DHR96 receptor are assayed in vivo by monitoring reporter gene expression.

Bioassays are set up as described in Example 6.1 to expose the insects to test compounds. The test flies used are obtained as described in Example 8.4, namely F1 flies comprising the transgenes for both the DHR96-GAL4 chimeric receptor and the reporter protein. Expression of the DHR96-GAL4 chimeric receptor is induced by an appropriate heatshock treatment so that the chimeric receptor protein is expressed and available for interaction with a test compound when it is applied in the bioassay.

The effects of test compounds on whole insects (larvae and adults) is monitored by measuring for any reporter gene expression, using appropriate visualisation equipment (e.g. using the Leica MZFLIII microscope with appropriate filters for detection of expression of the fluorescent reporter). Reporter gene expression is assessed over the duration of the experiment. Tissue samples are optionally isolated and assessed for reporter gene activation. Any other effects (e.g. morbidity) on the flies are also assessed. Compounds that cause reporter gene expression by this chimeric DHR96 receptor:UAS-reporter system are thus identified. Any background reporter gene expression is apparent in the control wells where no compound has been applied.

In this manner, compounds that bind to the ligand-binding domain of DHR96 and thereby elicit a conformational change resulting in reporter gene expression, are identifiable. Thus agonists of DHR96 which are capable of inducing a “detoxification response” in insects may be identified using this assay.

The skilled man will appreciate that the chimeric receptor and reporter gene constructs described above may equally be employed in a cell-based assay system as well as in the whole organism system described above. Such cell-based assay systems comprise yet a further embodiment of the invention.

### Example 9

### Cloning of dhr96 Homologues from other Insect Species

All the insect sequences available in public sequence databases that show significant similarity (≧50% identity over a match length>125 amino acids) to all or part of the DHR96 protein were identified. As of 4^{th} February 2005, the public database (Uniprot) identifiers for such insect sequences are:

| | |
|---|---|
| Drosphila melanogaster: | UNIPROT/Swissprot Q24143 |
| Drosophila grimshawi: | UniProt:Q01643 |
| Anopheles gambiae: | UniProt:Q7PQN6 |
| Bombyx mori: | Bmb040237 |

The Bombyx mori predicted protein sequence is obtained from the online supporting material for Silkworm genome publication: Draft Sequence for the Genome of the Domesticated Silkworm (Bombyx mori) Xia et al Science 10 Dec. 2004; 306: 1937-1940. These 4 insect sequences described above are used to generate a protein sequence alignment using the ClustalW alignment programme (Thompson, J. D., Higgins, D. G. and Gibson, T. J. 1994 “CLUSTAL W: improving the sensitivity of progressive multiple sequence alignment through sequence weighting, positions-specific gap penalties and weight matrix choice”, Nucleic Acids Research, 22:4673-4680), and visualised as a multiple sequence alignment file (data not shown).

The following primers were designed based on regions of amino acid sequence showing the greatest conservation between the four sequences:

| | | |
|---|---|---|
| Forward degenerate primer | (SEQ ID NO 20) | 5′ YTNYTNAARGGNGGNTGYACNGARATGATG 3′ |
| Reverse degenerate primer | (SEQ ID NO 21) | 5′ NCKNARNARRTARTARTARCTRTTYTGYTC 3′ |

A cDNA library is created for the insect species from which a dhr96 homologue is to be cloned, using standard methods (Sambrook, et al. 1989, Molecular Cloning, A Laboratory Manual, 2^{nd} Edition, Cold Spring Harbor Laboratory, New York).

Polymerase chain reactions (PCR) are carried out using the primers described above to amplify specific sequences from the cDNA library. Any DNA sequences thus amplified are sequenced to confirm homology to DHR96. The purified PCR products are then used as probes to isolate the relevant full-length cDNA from the cDNA library, using standard methods (Sambrook, et al. 1989, Molecular Cloning, A Laboratory Manual, ₂^{nd} Edition, Cold Spring Harbor Laboratory, New York). cDNAs thus identified are sequenced and compared to dhr96 to confirm that they are dhr96 homologues.

1. A transgenic insect or transgenic insect cell, wherein the level of expression of dhr96 or a homologue thereof has been reduced relative to the level of expression of dhr96 or said homologue in a non-transgenic insect or non-transgenic insect cell.

2. A transgenic insect or transgenic insect cell according to claim 1 wherein the homologue is from an insect selected from the group consisting of Drosophila sp., Bombyx mori, Tribolium castaneum, Aedes aegyptii, Anopheles gambiae, Anopheles albimanus, Anopheles stephensi, Ceratitis capitata, Pectinophora gossypiella, Helicoverpa zea, Bactrocera dorsalis, Anastrepha suspense, Musca domestica, Stomoxys calcitrans, and Lucilia cuprina.

3. A transgenic insect or transgenic insect cell according to claim 1 wherein the transgenic insect or transgenic insect cell is selected from the group of insects consisting of: Drosophila sp., Bombyx mori, Tribolium castaneum, Aedes aegyptii, Anopheles gambiae, Anopheles albimanus, Anopheles stephensi, Ceratitis capitata, Pectinophora gossypiella, Helicoverpa zea, Bactrocera dorsalis, Anastrepha suspense, Musca domestica, Stomoxys calcitrans, and Lucilia cuprina

4. A transgenic insect or transgenic insect cell according to claim 3 which is or comes from a species of Drosophila.

5. A transgenic insect or transgenic insect cell according to claim 4 which is or comes from D. melanogaster.

6. A transgenic insect or transgenic insect cell according to claim 1 wherein the level of expression of dhr96 or said homologue has been reduced through RNAi.

7. A transgenic insect or transgenic insect cell according to claim 1 wherein the level of expression of dhr96 or said homologue has been reduced through insertional mutagenesis.

8. A transgenic insect or transgenic insect cell according to claim 1 wherein the level of expression of dhr96 or said homologue has been reduced through homologous recombination.

9. A DNA construct comprising a first DNA sequence encoding a fragment of dhr96 or homologue thereof and a second DNA sequence which is the reverse complement of the first DNA sequence, wherein the first and second DNA sequences are present on the same strand of DNA and are operably linked to a promoter region and optionally a terminator region.

10. A DNA construct according to claim 9 wherein the first and second DNA sequences are separated by a third DNA sequence which acts a spacer sequence between the first and second DNA sequences.

11. A DNA construct according to claim 9 wherein the first DNA encodes a fragment of dhr96 from D. melanogaster.

12. A DNA construct according to claim 11 which comprises bases 58-661 of SEQ ID NO 1, or the reverse complement thereof.

13. A method of producing a transgenic insect comprising:
(i) transforming a first insect with a first DNA construct having a first DNA sequence encoding a fragment of dhr96 or homologue thereof and a second DNA sequence which is the reverse complement of the first DNA sequence wherein the first and second DNA sequences are present on the same strand of DNA and are operably linked to a promoter region and optionally a terminator region and wherein the promoter region has an inducible promoter;
(ii) transforming a second insect with a second DNA construct encoding a protein that is capable of inducing expression from the inducible promoter in the first DNA construct;
(iii) crossing the transformed insects resulting from steps (i) and (ii) to obtain progeny;
wherein a population of the progeny resulting from step (iii) comprises both the first and the second DNA constructs and when the protein encoded in the second DNA construct is expressed, the level of expression of dhr96 or said homologue is reduced in said population of the progeny relative to the level of expression of dhr96 or said homologue in a non-transgenic insect.

14. A method of producing a transgenic insect comprising:
(i) transforming a first insect with a first DNA construct having a first DNA sequence encoding a fragment of dhr96 or homoloque thereof and a second DNA sequence which is the reverse complement of the first DNA sequence wherein the first and second DNA sequences are present on the same strand of DNA and are operably linked to a promoter region and optionally a terminator region, and wherein the promoter region has an inducible promoter; and,
(ii) crossing the transformed insect resulting from step (i) with an insect that expresses an inducing protein that is capable of inducing expression from the inducible promoter in the first DNA construct, to obtain progeny;
wherein a population of the progeny resulting from step (ii) comprise the first DNA construct and express the inducing protein so that the level of expression of dhr96 or said homologue is reduced in said population of the progeny relative to the level of expression of dhr96 or said homologue in a non-transgenic insect.

15. A method of identifying or verifying the ability of a compound to act as an insecticide, which comprises:
(i) placing a transgenic insect or transgenic insect cell in contact with the compound;
(ii) assessing the transgenic insect or transgenic insect cell that has been placed in contact with the compound in step (i) for any deleterious effect on the transgenic insect or transgenic insect cell;
wherein the presence of a deleterious effect is indicative that said compound is capable of acting as an insecticide.

16. A method of identifying the ability of a compound to act as a pro-insecticide, which comprises:
(i) placing a transgenic insect or transgenic insect cell in contact with the compound;
(ii) assessing said transgenic insect or transgenic insect cell that has been placed in contact with the compound in step (i) for any deleterious effect on the transgenic insect or transgenic insect cell; and
(iii) comparing the effects observed in step (ii) to the effects observed on a) a non-transgenic insect or non-transgenic insect cell and/or b) a transgenic insect or transgenic insect cell wherein the level of expression of dhr96 or homologue thereof has been increased in comparison to the level of expression in a non-transgenic insect or non-transgenic insect cell;
wherein the presence of a deleterious effect on a) a non-transgenic insect or non-transgenic insect cell and/or b) a transgenic insect or transgenic insect cell wherein the level of expression of dhr96 or homologue thereof has been increased in comparison to the level of expression in a non-transgenic insect or non-transgenic insect cell coupled to the absence of a deleterious effect on transgenic insect or transgenic insect cell in step (ii) is indicative that said compound is capable of acting as a pro-insecticide.

17. A method of identifying a compound that regulates expression of dhr96 or a homologue thereof, which comprises:
(i) culturing in a suitable growth medium a population of cells transformed with a DNA construct comprising a reporter gene operably linked to the promoter region of dhr96 or a homologue thereof
(ii) incubating said compound with a population of cells according to step (i);
(iii) determining the level of expression of said reporter gene in cells that have been incubated with said compound; and
(iv) determining the level of expression of said reporter gene in a population of cells according to step (i);
wherein, a difference in the levels of reporter gene expression determined at (iii) and
(iv) is indicative that said compound is capable of acting as a regulator of expression of said insect nuclear hormone receptor gene.

18. A method of identifying a compound that regulates expression of dhr96 or homologue thereof, which comprises:
(i) culturing on a suitable growth medium an insect transformed with a DNA construct comprising a reporter gene operably linked to the promoter region of dhr96 or a homologue thereof
(ii) incubating said compound with transformed insect as described in step (i);
(iii) determining the level of expression of said reporter gene in the transformed insect of step (ii); and
(iv) determining the level of expression of said reporter gene in a transformed insect as described in step (i);
wherein, a difference in the levels of reporter gene expression determined at (iii) and (iv) is indicative that said compound is capable of acting as a regulator of expression of said insect nuclear hormone receptor gene.

19. A DNA construct comprising a first DNA sequence encoding the ligand binding domain of DHR96 and a second DNA sequence encoding a heterologous eukaryotic DNA binding domain, operably linked to a promoter and optionally a terminator region.

20. A DNA construct according to claim 19, wherein the first DNA sequence additionally encodes the hinge region and the F domain of DHR96.

21. A DNA construct according to claim 20, wherein the first DNA sequence encodes a peptide having the amino acid sequence given in SEQ ID NO 15.

22. A DNA construct according to claim 19, wherein the heterologous eukaryotic DNA binding domain is the GAL4 DNA binding domain.

23. A DNA construct according to claim 19, wherein the promoter is the hsp70 promoter sequence.

24. A transgenic insect or transgenic insect cell comprising a first DNA construct according to claim 22, and a second DNA construct, said second DNA construct comprising a GAL4 responsive element situated upstream of and linked to a reporter gene.

25. A transgenic insect or transgenic insect cell according to claim 24 wherein said reporter gene is a fluorescent reporter gene selected from the group consisting of amfp486, asfp595, zfp538 and zfp506.

26. A method of identifying the ability of a compound to act as a regulator of DHR96 function, which comprises:
(i) contacting a transgenic insect or transgenic insect cell with the compound; and
(ii) measuring the level of reporter gene expression in said transgenic insect or transgenic insect cell,
wherein the level of reporter gene expression correlates with the ability of the compound to regulate DHR96 function.

27. A transgenic insect or transgenic insect cell of a first insect species comprising a nuclear hormone receptor gene from a second insect species, wherein said nuclear hormone receptor gene is dhr96 or a homologue thereof.
